(19) United States
(12) Patent Application Publication
(10) Pub. No.: US 2013/0171150 A1
(43) Pub. Date: Jul. 4, 2013

(54) FELINE PICORNA VIRUS AND USES THEREOF

(71) Applicants: Amit Kapoor, New York, NY (US); Edward J. Dubovi, Ithaca, NY (US); W. Ian Lipkin, New York, NY (US)

(72) Inventors: Amit Kapoor, New York, NY (US); Edward J. Dubovi, Ithaca, NY (US); W. Ian Lipkin, New York, NY (US)

(21) Appl. No.: 13/553,461

(22) Filed: Jul. 19, 2012

Related U.S. Application Data
(60) Provisional application No. 61/509,245, filed on Jul. 19, 2011.

(51) Int. Cl. C07K 14/085; G01N 33/569; C07K 16/10; C12Q 1/70; C12N 7/00; C12N 7/04

(52) U.S. Cl. CPC C07K 14/085; C12N 7/00; C12N 7/04; C07K 16/1009; C12Q 1/701; G01N 33/56983
USPC 424/139.1; 536/23.72; 435/302.1; 530/350; 435/235.1; 435/236; 530/387.9; 424/216.1; 424/186.1; 536/24.33; 536/24.32; 435/5

(57) ABSTRACT

The invention is directed to a Feline Picorna Virus, an isolated nucleic acid and amino acid sequences therefrom, and uses thereof.

The content of all patent applications, published patents applications, issued and granted patents, and all references cited in this application are hereby incorporated by reference.

### BACKGROUND

Picornaviruses are non-enveloped, positive-stranded RNA viruses with an icosahedral capsid. Picornaviruses are separated into 12 distinct genera and include many important pathogens of humans and animals. The diseases they cause are varied, ranging from acute “common-cold”-like illnesses, to poliomyelitis, to chronic infections in livestock. Picornaviruses comprise the genera Aphthovirus, Avihepatovirus, Cardiovirus, Enterovirus, Erbovirus, Hepatovirus, Kobuvirus, Parechovirus, Sapelovirus, Senecavirus, Teschovirus, and Tremovirus. The present invention provides an isolated feline picorna virus (FeSV) and uses thereof.

### SUMMARY

This invention describes the first sequence information of a highly divergent picornavirus species in cats suffering with multiple organ failure and wasting diseases. It is likely this new picornavirus to be a pathogen not only in cats, but also in dogs and other mammalian species. The reported virus species belongs to family Picornaviridae. The invention provides the complete nucleotide sequence, translated protein sequence of this new virus named Feline Picornavirus/Sapelovirus (FeSV). The phylogenetic analysis done using nucleotide and protein alignments confirms FeSV as unique and highly divergent to any other known picornavirus reported to date. This virus is the first picornavirus known to infect and cause diseases in cats and dogs (feline and canine host).

Phylogenetically FeSV is distantly related to human and simian enteroviruses. FeSV showed only <50% protein identity to any known picornavirus reported so far.

In certain aspects the invention provides an isolated feline picorna virus and uses thereof.

In certain aspect the invention provides nucleic and amino acids sequences, antigens derived from the feline picorna virus, immunogenic compositions comprising antigens from the feline picorna virus, antibodies binding to antigens from the feline picorna virus, immunoassays, and nucleic acid assays for detection of the FeSV pathogen in subject animals.

An immunogenic composition or vaccine, and method of treatment are provided by the present invention. The immunogenic composition is useful for treating, preventing, or lessening the severity of clinical symptoms associated with disease-causing organisms in cats, dogs or other mammals susceptible to the feline picorna virus described herein, utilizing immunogenic compositions. Immunogenic compositions may comprise a whole virus as described herein, for example inactivated virus and/or antigen(s) from the Feline picorna virus described herein, or a feline picorna virus component, and a pharmaceutically acceptable carrier. In non-limiting embodiments a “feline picorna virus component” refers to any structural part of a virus, such as protein, peptide, other structures, nucleic acid, or other proteins or nucleic acids coded by the virus genome and produced during the virus replication, or any part of the above-mentioned component. The nucleic acid encompassed by the term may be DNA or RNA coding for the entire virus or a negative strand corresponding to the virus RNA, or a fragment of said DNA or RNA molecule.

In certain aspects the invention provides an isolated nucleic acid comprising, consisting essentially of, or consisting of SEQ ID NO: 1, or an isolated nucleic acid represented by SEQ ID NO: 1. In certain aspects, the invention provides an isolated nucleic acid comprising, consisting essentially of, or consisting of from 10 to 7496 consecutive nucleotides having a sequence selected from: SEQ ID NO: 1, a sequence complementary to SEQ ID NO: 1, a sequence having about 85% identity to SEQ ID NO: 1, or a sequence having about 85% identity to a sequence complementary to SEQ ID NO: 1, wherein the % identity is determined by analysis with a sequence comparison algorithm. Picornaviruses are highly diverse viruses, and the invention provides an isolated nucleic acid comprising, consisting essentially of, or consisting of from 10 to 7496 consecutive nucleotides having a sequence selected from: SEQ ID NO: 1, a sequence complementary to SEQ ID NO: 1, a sequence having about 25-35% (65 to 75% identity to SEQ ID NO: 1, or a sequence having about 25-35% (65 to 75% identity to a sequence complementary to SEQ ID NO: 1, wherein the % identity is determined by analysis with a sequence comparison algorithm.

In certain aspects the invention provides an isolated nucleic acid comprising, consisting essentially of, or consisting of a nucleic acid encoding any one of the proteins of SEQ ID NOs: 2-18, or an isolated nucleic acid comprising a nucleic acid encoding a conserved variant of any one of the proteins of SEQ ID NO: 2-18. In certain aspects the invention provides an isolated nucleic acid comprising, consisting essentially of, or consisting of a degenerate nucleic acid encoding any one of the proteins of SEQ ID NOs: 2-18, or an isolated nucleic acid comprising a degenerate nucleic acid encoding a conserved variant of any one of the proteins of SEQ ID Nos: 2-18.

In certain aspects the invention provides an isolated feline picorna virus (FeSV) comprising a nucleic acid encoding any one of the proteins of SEQ ID NO: 2-18, or a variant thereof, for example but not limited to a conserved variant.

In certain aspects the invention provides a replicable vector comprising, or consisting essentially of any one of the nucleic acids of the invention, including but not limited to nucleic acids encoding the peptides of the invention.

In certain aspects the invention provides an isolated peptide comprising, consisting essentially of, or consisting of any on of the peptides of SEQ ID NOs: 2-18, or a conserved variant of SEQ ID NOs: 2-18. In certain aspects the invention provides an isolated peptide represented by SEQ ID NOs: 2-18, or a conserved variant of SEQ ID NOs: 2-18. In certain aspects, the invention provides a composition comprising the inventive peptides. In certain embodiments, the composition is immunogenic. A skilled artisan can readily determine the immunogenicity of the inventive peptides or components of FeSV.

In certain aspects the invention provides an immunogenic composition comprising, consisting essentially of, or consisting of FeSV, for example whole FeSV, a component of FeSV, or a combination thereof. In non-limiting embodiments, the component is a nucleic acid of FeSV or a fragment thereof, or a peptide of FeSV, or a fragment thereof. In non-limiting embodiments, the whole FeSV is attenuated, inactivated, or a combination thereof.

In certain aspects the invention provides a pharmaceutical or veterinary composition for the treatment of a feline picorna virus infection or symptoms thereof, comprising, consisting essentially of, or consisting of an immunogenic composition comprising, consisting essentially of, or consisting of FeSV, an immunogenic composition comprising, consisting essentially of, consisting of a component of FeSV, or a combination thereof. A skilled artisan can readily determine the immunogenicity of components of FeSV. In certain aspects the invention provides a pharmaceutical or veterinary composition for the treatment of a feline picorna virus infection or symptoms thereof, comprising, consisting essentially of, or consisting of an immunogenic composition comprising, consisting essentially of, or consisting of an antibody against FeSV, an antibody against a component of FeSV, or a combination thereof.

In certain aspects the invention provides a method to treat, prevent or reduce the severity of a feline picorna virus infection or symptoms thereof, comprising, consisting essentially of, or consisting of administering a therapeutically effective amount of the pharmaceutical composition of the invention.

In certain aspects the invention provides an isolated nucleic acid comprising, consisting essentially of, or consisting of 10 to 30 consecutive nucleotides selected from SEQ ID NO: 1, or a sequence complementary to SEQ ID NO: 1. In certain aspects the invention provides an isolated nucleic acid comprising, consisting essentially of, or consisting of 10 to 30 consecutive nucleotides selected from SEQ ID NO: 19, positions 1 to 372 of SEQ ID NO: 1, which is the 5′UTR of SEQ ID NO: 1, SEQ ID NO: 20, positions 2962 to 7494 of SEQ ID NO: 1, SEQ ID NO: 21, positions 6007 to 7389 of SEQ ID NO: 1, or a sequence complementary to SEQ ID NOs: 19, 20, or 21. In certain aspects, the invention provides a composition comprising, consisting essentially of, or consisting of the inventive nucleic acids, primers and probes. In certain aspects, the invention provides a kit comprising at least one isolated nucleic acid of the invention and instructions for use. In certain embodiments, the kit optionally comprises containers for sample collection, reagents which are suitable as controls, for example a nucleic acid which can serve as a positive control, and/or a nucleic acid which can serve as a negative control, reagents such as reaction buffers and/or mixes, enzyme, and the like. In certain embodiments, the nucleic aids are lyophilized.

In certain aspects, the invention provides an antibody that specifically binds to an epitope comprised in FeSV, wherein FeSV is encoded by SEQ ID NO: 1 or a degenerate variant of SEQ ID NO: 1, or the antibody binds to an epitope comprised in a component of the FeSV encoded by SEQ ID NO:1 or a degenerate variant of SEQ ID NO: 1. In certain aspects, the invention provides an antibody that specifically binds to any one of the peptides of SEQ ID NOs:2-18, or a any combination thereof, or a fragment thereof. In certain aspects the invention provides an antibody which binds to an epitope comprised in one or more of VP1 (SEQ ID NO: ______), VP2, (SEQ ID NO: ______), VP3 (SEQ ID NO: ______), or VP4 (SEQ ID NO:______) of FeSV. In non-limiting examples the antibody is an isolated antibody. In certain embodiments, the antibody is monoclonal antibody. In certain embodiments, the antibody is a polyclonal antibody. In certain embodiments, the antibodies are conjugated to various agents which facilitate use of the antibodies in immuno-detection assays. In certain embodiments the epitope is immunogenic. In certain embodiments the epitope is antigenic.

In certain aspects, the invention provides a kit comprising an antibody of the invention and instructions for use. In certain embodiments, the kit optionally comprises containers for sample collection, reagents which are suitable as controls, for example polypeptide(s) which can serve as a positive control, and/or polypeptide(s) which can serve as a negative control, reagents such as reaction buffers and/or mixes, enzyme, and the like. In certain embodiments, the nucleic aids are lyophilized.

In certain aspects, the invention provides a method to detect FeSV in a biological sample, the method comprising determining the presence or absence in a biological sample from a subject in need thereof of: FeSV, a component of FeSV, an antibody that specifically binds to an epitope comprised within FeSV, or an antibody that specifically binds to an epitope comprised in a component of FeSV or an epitope comprised within any one of SEQ ID NOs:2-18, or any combination thereof. In certain embodiments, determining is carried out by PCR, for example but not limited to real time qPCR, or RT-PCR, immunodetection, immunohistochemistry, in situ hybridization, Nucleic acid sequence based amplification (NASBA) method, by isolating or growing FeSV in cell culture, any other suitable method, or any combination thereof. In certain embodiments, the biological sample is from a cat, a dog, or humans.

In certain aspects, the invention provides a method for determining the presence or absence of FeSV in a biological sample, the method comprising: a) contacting nucleic acid from a biological sample with at least one primer which is a nucleic acid of the invention; b) subjecting the nucleic acid and the primer to amplification conditions, and c) determining the presence or absence of amplification product, wherein the presence of amplification product indicates the presence of RNA associated with FeSV in the sample.

In certain aspects, the invention provides a method for determining the presence or absence of FeSV in a biological sample, the method comprising: a) contacting a biological sample with an antibody that specifically binds to a FeSV encoded by SEQ ID NO:1; VP1, VP2, VP3 or VP4 polypeptide encoded by SEQ ID NO:1; or any combination thereof, and b) determining whether or not the antibody binds to an epitope in the biological sample, wherein binding indicates that the biological sample contains FeSV. In certain embodiments, the determining comprises use of a lateral flow assay or ELISA. In certain embodiments, the determining comprises determining whether the antibodies are IgM antibodies, wherein detection of IgM antibodies is indicative of a recent infection of the sample by a picornavirus FeSV.

In certain embodiment, the methods and compositions of the invention are suitable for veterinary or pharmaceutical applications.

### BRIEF DESCRIPTION OF FIGURES

FIGS. 1A and 1B show genomic annotation of Feline PicornaVirus/Sapelovirus (FeSV). Abbreviations: FeSV—feline sapelovirus, SV—Sapelovirus, EV—Enterovirus and FIRV—human rhinovirus. FIG. 1A shows predicted genome organization of FeSV, showing amino acid and nucleotide positions of predicted cleavage sites in the polyprotein (numbering based on the FeSV genomic sequence). The numbers on top of the genome organization/annotation refer to nucleic acid positions of SEQ ID NO: 1. The numbers on the bottom of the genome organization/annotation refer to amino acid positions of SEQ ID NO: 2. Sites were predicted by NetPicoRNA analysis and by alignment with known cleavage sites annotated in the sapelovirus sequence, accession number AF406813. The position of the VP1/2A cleavage site Q/N predicted for amino acid position 864 is speculative and does not align exactly with the corresponding site in AF406813 at position 878 (Q/T). Peptide L corresponds SEQ ID NO: 3 (which includes amino acid positions 1-64 of SEQ ID NO: 2). Peptide VP4 corresponds to SEQ ID NO: 4 (which includes amino acid positions 65-114 of SEQ ID NO: 2). Peptide VP2 corresponds to SEQ ID NO: 5 (which includes amino acid positions 115-354 of SEQ ID NO: 2). Peptide VP3 corresponds to SEQ ID NO: 6 (which includes amino acid positions 355-582 of SEQ ID NO: 2). Peptide VP1 corresponds to SEQ ID NO: 7 (which includes amino acid positions 583-863 of SEQ ID NO: 2). SEQ ID NO: 8 is a P1 propeptide (which includes amino acid positions 65-863 of SEQ ID NO: 2). SEQ ID NO: 9 is VP propeptide (which includes amino acid positions 65-354 of SEQ ID NO: 2). SEQ ID NO: 10 is VP propeptide (which includes amino acid positions 66-582 of SEQ ID NO: 2). SEQ ID NO:11 is VP propeptide (which includes amino acid positions 355-863 of SEQ ID NO: 2). SEQ ID NO: 12 is 2A protein (which includes amino acid positions 864-1087 of SEQ ID NO: 2). SEQ ID NO: 13 is 2B protein (which includes amino acid positions 1088-1190 of SEQ ID NO: 2). SEQ ID NO: 14 is 2C protein (which includes amino acid positions 1191-1526 of SEQ ID NO: 2). SEQ ID NO: 15 is 3A protein (which includes amino acid positions 1527-1673 of SEQ ID NO: 2). SEQ ID NO: 16 is 3B protein (which includes amino acid positions 1674-1696 of SEQ ID NO: 2). SEQ ID NO: 17 is 3C protein (which includes amino acid positions 1697-1878 of SEQ ID NO: 2). SEQ ID NO: 18 is 3D protein (polymerase) (which includes amino acid positions 1879-2337 of SEQ ID NO: 2). FIG. 1B shows mean divergence of FeSV translated amino acid sequences from other sapeloviruses and examples from the Enterovirus genus (EV species A and HRV species A). The leader-encoding sequences of sapeloviruses were omitted from the divergence scan because they could not be aligned satisfactorily with each other.

FIG. 2 shows phylogenetic analysis of Feline Sapelovirus. Phylogenetic analysis of translated amino acid sequences from the P1 and P3 regions of Feline Sapelovirus (picornavirus) using neighbor-joining of Poisson-corrected pairwise distances. The tree includes available sapelovirus complete genome sequences and representative sequences (up to 4) of all known Enterovirus species. The trees were rooted using the FMDV sequence, NC_{—}01 1450 as an outgroup. Data were bootstrap re-sampled 100 times with values shown on branches.

FIG. 3 shows the complete nucleic acid sequence (SEQ ID NO: 1) of the FeSV virus.

FIG. 4 shows the complete amino acid sequence (SEQ ID NO: 2) of the FeSV virus.

### DETAILED DESCRIPTION

### Nucleic and Amino Acids

The present invention provides picornavirus nucleic acid sequences. These nucleic acid sequences may be useful for, inter alia, expression of picornavirus-encoded proteins or fragments, variants, or derivatives thereof, generation of antibodies against picornavirus proteins, generation of primers and probes for detecting picornaviruses and/or for diagnosing picornavirus infection, generating vaccines against picornaviruses, and screening for drugs effective against picornaviruses, as described below.

In certain aspects, the invention is directed to an isolated nucleic acid sequence as provided in SEQ ID NO: 1. The invention is directed to nucleic acid sequences encoding the peptides of SEQ ID NOs: 2-18. A skilled artisan appreciates that due to the degeneracy of the nucleic acid code, the peptides of SEQ ID NOS: 2-18 can be encoded by more than one nucleic acids. The invention provides these degenerate nucleic acid sequences which encode peptides of SEQ ID NOs: 2-18. The invention is directed to an isolated nucleic acid complementary to SEQ ID NO: 1. The invention is directed to a fragment of SEQ ID NO 1, for example a fragment of SEQ ID NO: 1, or a variant, which encodes a peptide of SEQ ID NO: 2-18.

In certain aspects, the invention is directed to isolated nucleic acid sequence variants of SEQ ID NO: 1. In certain aspects, the invention is directed to isolated nucleic acid sequence variant which is a fragment of SEQ ID NO 1, for example a fragment of SEQ ID NO: 1, or a variant, which encodes any one of the peptides of SEQ ID NO: 2-18. Contemplated variants include but are not limited to nucleic acid sequences having at least from about 50% to about 55% identity. Contemplated variants include but are not limited to nucleic acid sequences having at least from about 55.1% to about 60% identity. Contemplated variants include but are not limited to nucleic acid sequences having at least from about 60.1% to about 65% identity. Contemplated variants include but are not limited to nucleic acid sequences having at least from about 65.1% to about 70% identity. Contemplated variants include but are not limited to nucleic acid sequences having at least from about 70.1% to about 75% identity. Contemplated variants include but are not limited to nucleic acid sequences having at least from about 75.1% to about 80% identity. Contemplated variants include but are not limited to nucleic acid sequences having at least from about 80.1% to about 85% identity. Contemplated variants include but are not limited to nucleic acid sequences having at least from about 85.1% to about 90% identity. Contemplated variants include but are not limited to nucleic acid sequences having at least from about 90.1% to about 95% identity. Contemplated variants include but are not limited to nucleic acid sequences having at least from about 95.1% to about 97% identity. Contemplated variants include but are not limited to nucleic acid sequences having at least from about 97.1% to about 99% identity. Programs and algorithms for sequence alignment and comparison of % identity and/or homology between nucleic acid sequences, or polypeptides, are well known in the art, and include BLAST, SIM alignment tool, and so forth.

The invention is directed to an isolated nucleic acid sequence comprising from about 10 to about 50 consecutive nucleotides from any one of SEQ ID NO: 1 or a sequence complementary to SEQ ID NO: 1. The invention is directed to an isolated nucleic acid sequence comprising from about 10 to about 100 consecutive nucleotides from any one of SEQ ID NO: 1 or a sequence complementary SEQ ID NO: 1. The invention is directed to an isolated nucleic acid sequence comprising from about 10 to about 200 consecutive nucleotides from any one of SEQ ID NO: 1 or a sequence complementary to SEQ ID NO: 1. The invention is directed to an isolated nucleic acid sequence comprising from about 10 to about 300 consecutive nucleotides from any one of SEQ ID NO: 1 or a sequence complementary to SEQ ID NO: 1. The invention is directed to an isolated nucleic acid sequence comprising from about 10 to about 400 consecutive nucleotides from SEQ ID NO: 1 or a sequence complementary to SEQ ID NO: 1. The invention is directed to an isolated nucleic acid sequence comprising from about 10 to about 500 consecutive nucleotides from any one of SEQ ID NO: 1 or a sequence complementary to SEQ ID NO: 1. The invention is directed to an isolated nucleic acid sequence comprising from about 10 to about 1000 consecutive nucleotides from any one of SEQ ID NO: 1 or a sequence complementary to SEQ ID NO: 1. The invention is directed to an isolated nucleic acid sequence comprising from about 10 to about 1400 consecutive nucleotides from any one of SEQ ID NO: 1 or a sequence complementary to SEQ ID NO: 1. The invention is directed to an isolated nucleic acid sequence comprising from about 10 to about 2000 consecutive nucleotides from any one of SEQ ID NO: 1 or a sequence complementary to SEQ ID NO: 1. The invention is directed to an isolated nucleic acid sequence comprising from about 10 to about 2400 consecutive nucleotides from any one of SEQ ID NO: 1 or a sequence complementary to SEQ ID NO: 1. The invention is directed to an isolated nucleic acid sequence comprising from about 10 to about 2700 consecutive nucleotides from any one of SEQ ID NO: 1 or a sequence complementary to SEQ ID NO: 1. The invention is directed to an isolated nucleic acid sequence comprising from about 10 to about 2900 consecutive nucleotides from any one of SEQ ID NO: 1 or a sequence complementary to SEQ ID NO: 1. The invention is directed to an isolated nucleic acid sequence comprising from about 10 to about 3100 consecutive nucleotides from any one of SEQ ID NO: 1 or a sequence complementary to SEQ ID NO:1. The invention is directed to an isolated nucleic acid sequence comprising from about 10 to about 3500 consecutive nucleotides from any one of SEQ ID NO: 1 or a sequence complementary to SEQ ID NO: 1. The invention is directed to an isolated nucleic acid sequence comprising from about 10 to about 3700 consecutive nucleotides from any one of SEQ ID NO: 1 or a sequence complementary to SEQ ID NO: 1. The invention is directed to an isolated nucleic acid sequence comprising from about 10 to about 4000 consecutive nucleotides from any one of SEQ ID NO: 1 or a sequence complementary to SEQ ID NO: 1. The invention is directed to an isolated nucleic acid sequence comprising from about 10 to about 4500 consecutive nucleotides from any one of SEQ ID NO: 1 or a sequence complementary to SEQ ID NO: 1. The invention is directed to an isolated nucleic acid sequence comprising from about 10 to about 5000 consecutive nucleotides from any one of SEQ ID NO: 1 or a sequence complementary to SEQ ID NO: 1. The invention is directed to an isolated nucleic acid sequence comprising from about 10 to about 5500 consecutive nucleotides from any one of SEQ ID NO: 1 or a sequence complementary to SEQ ID NO: 1. The invention is directed to an isolated nucleic acid sequence comprising from about 10 to about 6000 consecutive nucleotides from any one of SEQ ID NO: 1 or a sequence complementary to SEQ ID NO: 1. The invention is directed to an isolated nucleic acid sequence comprising from about 10 to about 6500 consecutive nucleotides from any one of SEQ ID NO: 1 or a sequence complementary to SEQ ID NO: 1. The invention is directed to an isolated nucleic acid sequence comprising from about 10 to about 7000 consecutive nucleotides from any one of SEQ ID NO: 1 or a sequence complementary to SEQ ID NO: 1. The invention is directed to an isolated nucleic acid sequence comprising from about 10 to about 7500 consecutive nucleotides from any one of SEQ ID NO: 1 or a sequence complementary to SEQ ID NO: 1.

In other aspects the invention is directed to isolated nucleic acid sequences such as primers and probes, comprising nucleic acid sequences derived from SEQ ID NO: 1. Such primers and/or probes may be useful for detecting the presence of the picornavirus of the invention, for example in samples of bodily fluids such as blood, saliva, or urine, or fecal sample from a subject, and thus may be useful in the diagnosis of picornavirus infection. Such probes can detect polynucleotides of SEQ ID NO: 1 in samples which comprise picornaviruses represented by SEQ ID NO: 1. The isolated nucleic acids which can be used as primer and/probes are of sufficient length to allow hybridization with, i.e. formation of duplex with a corresponding target nucleic acid sequence, a nucleic acid sequences of any one of SEQ ID NO: 1, or a variant thereof.

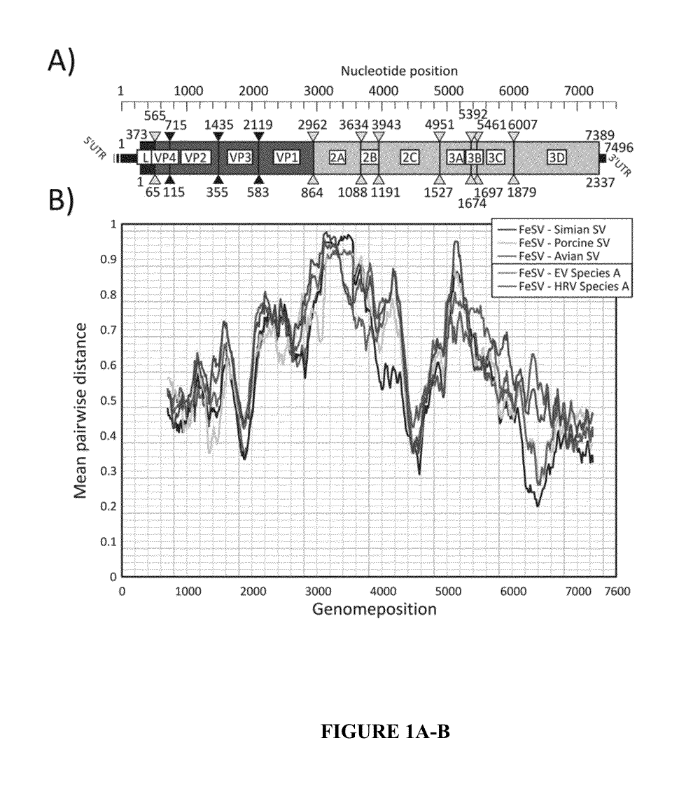

The isolated nucleic acid of the invention which can be used as primers and/or probes can comprise about 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 consecutive nucleotides from any one of SEQ ID NO: 1-23, or sequences complementary to SEQ ID NO: 1. The isolated nucleic acid of the invention which can be used as primers and/or probes can comprise from about 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 and up to about 20, 25, 30, 35, 40, 45, 50, 55, 60, 65, 70, 75, 80, 85, 90, 95 and 100 consecutive nucleotides from any one of SEQ ID NO: 1, or sequences complementary to SEQ ID NO: 1. The isolated nucleic acid of the invention which can be used as primers and/or probes can comprise from 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 and up to 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95 and 100 consecutive nucleotides from any one of SEQ ID NO: 1, or sequences complementary to SEQ ID NO: 1. The isolated nucleic acid of the invention which can be used as primers and/or probes can comprise 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95 and 100 consecutive nucleotides from any one of SEQ ID NO: 1, or sequences complementary to SEQ ID NO: 1. In certain embodiments, the primers and probes are suitable for diagnostic detection of the FeSV from a biological sample. In certain embodiments, for diagnostic detection of the FeSV, for example through PCR, probes and/primers are derived from conserved regions in the genome of FeSV. Non-limiting examples of conserved regions with the FeSV genome are the 5′UTR (SEQ ID NO: 19, which corresponds to positions 1-373 of SEQ ID NO:1), sequences encoding non-structural proteins, for example non-structural proteins 2A, 2B, 2C, 3A, 3B, 3C and 3D in FIG. 1. In certain embodiments, the primers and/probes of the invention exclude nucleic acids encoding VPs as shown in FIG. 1.

The invention is also directed to primer and/or probes which can be labeled by any suitable molecule and/or label known in the art, for example but not limited to fluorescent tags suitable for use in Real Time PCR amplification, for example TaqMan™, cybergreen, TAMRA and/or FAM probes; radiolabels, and so forth. In certain embodiments, the oligonucleotide primers and/or probe further comprises a detectable non-isotopic label selected from the group consisting of: a fluorescent molecule, a chemiluminescent molecule, an enzyme, a cofactor, an enzyme substrate, and a hapten.

In certain aspects, the invention is directed to primer sets comprising isolated nucleic acids as described herein, which primer set are suitable for amplification of nucleic acids from samples which comprises picornaviruses represented by any one of SEQ ID NO: 1, or variants thereof. Primer sets can comprise any suitable combination of primers which would allow amplification of a target nucleic acid sequences in a sample which comprises picornaviruses represented by any one of SEQ ID NO: 1, fragments or variants thereof. Amplification can be performed by any suitable method known in the art, for example but not limited to PCR, RT-PCR, transcription mediated amplification (TMA).

For example, the nucleic acids described herein represented by any one of SEQ ID NO: 1, fragments or variants thereof can be used with any method described herein suitable for detecting the presence or absence of the novel picornavirus in a biological sample. In one embodiment, the method can comprise contacting nucleic acid from a biological sample with at least one primer which is a synthetic nucleic acid which has a sequence consisting of from about 10 to about 30 consecutive nucleotides from a nucleic acids sequence selected from the group of sequences consisting of SEQ ID NO: 1, subjecting the nucleic acid and the primer to amplification conditions, and determining the presence or absence of amplification product, wherein the presence of amplification product indicates the presence of RNA associated with picornavirus in the sample. For example, the nucleic acids described herein are suitable for detecting the presence or absence of picornaviruses in a sample, for example, see Briese et al., 2008; Dominguez et al., 2008 and Renwick et al., 2007—each of which is incorporated in their entirety and any sequences cited therein are incorporated by reference to the same extent as if each was specifically and individually indicated to be incorporated by reference.

The scope of the present invention is not limited to the exact sequence of the nucleotide sequences disclosed herein, or the amino acid sequences disclosed herein, or the use thereof. The invention contemplates certain modifications to the sequence, including deletions, insertions, and substitutions, that are well known to those skilled in the art as well as functional equivalents thereof.

A person of ordinary skill in the art recognizes that due to the redundancy of the genetic code, different codons encode the same amino acid. In certain aspects, the invention provides a nucleic acid which is a degenerate variant of SEQ ID NO: 1.

Hybridization Conditions

As used herein, the phrase “stringent hybridization conditions” refers to conditions under which a probe, primer or oligonucleotide will hybridize to its target sequence, and can hybridize, for example but not limited to, variants of the disclosed polynucleotide sequences, including allelic or splice variants, or sequences that encode orthologs or paralogs of presently disclosed polypeptides. The precise conditions for stringent hybridization are typically sequence-dependent and will be different in different circumstances. Longer sequences hybridize specifically at higher temperatures than shorter sequences. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Nucleic acid hybridization methods are disclosed in detail by Kashima et al. (1985) Nature 313:402-404, and Sambrook et al. (1989) Molecular Cloning: A Laboratory Manual, 2nd Ed., Cold Spring Harbor Laboratory, Cold Spring Harbor, N.Y (“Sambrook”); and by Haymes et al., “Nucleic Acid Hybridization: A Practical Approach”, IRL Press, Washington, D.C. (1985), which references are incorporated herein by reference.

In general, stringency is determined by the temperature, ionic strength, and concentration of denaturing agents (e.g., formamide) used in a hybridization and washing procedure. The degree to which two nucleic acids hybridize under various conditions of stringency is correlated with the extent of their similarity. Numerous variations are possible in the conditions and means by which nucleic acid hybridization can be performed to isolate nucleic sequences having similarity to the nucleic acid sequences known in the art and are not limited to those explicitly disclosed herein. Such an approach may be used to isolate polynucleotide sequences having various degrees of similarity with disclosed nucleic acid sequences, such as, for example, nucleic acid sequences having 60% identity, or about 70% identity, or about 80% or greater identity with disclosed nucleic acid sequences.

Stringent conditions are known to those skilled in the art and can be found in Current Protocols In Molecular Biology, John Wiley & Sons, N.Y. (1989), 6.3.1-6.3.6. In certain embodiments, the conditions are such that sequences at least about 65%, 70%, 75%, 85%, 90%, 95%, 98%, or 99% homologous to each other typically remain hybridized to each other. A non-limiting example of stringent hybridization conditions is hybridization in a high salt buffer comprising 6× sodium chloride/sodium citrate (SSC), 50 mM Tris-HC1 (pH 7.5), 1 nM EDTA, 0.02% PVP, 0.02% Ficoll, 0.02% BSA, and 500 mg/ml denatured salmon sperm DNA at 65° C. This hybridization is followed by one or more washes in 0.2×SSC, 0.01% BSA at 50° C. Another non-limiting example of stringent hybridization conditions are hybridization in 6× sodium chloride/sodium citrate (SSC) at about 45° C., followed by one or more washes in 0.2×SSC, 0.1% SDS at 50-65° C. Examples of moderate to low stringency hybridization conditions are well known in the art.

Polynucleotides homologous to the sequences illustrated in the Sequence Listing and figures can be identified, e.g., by hybridization to each other under stringent or under highly stringent conditions. Single stranded polynucleotides hybridize when they associate based on a variety of well characterized physical-chemical forces, such as hydrogen bonding, solvent exclusion, base stacking and the like. The stringency of a hybridization reflects the degree of sequence identity of the nucleic acids involved, such that the higher the stringency, the more similar are the two polynucleotide strands. Stringency is influenced by a variety of factors, including temperature, salt concentration and composition, organic and non-organic additives, solvents, etc. present in both the hybridization and wash solutions and incubations (and number thereof, as described in more detail in the references cited above.

Encompassed by the invention are polynucleotide sequences that are capable of hybridizing to the claimed polynucleotide sequences, including any of the nucleic acid sequences disclosed herein, and fragments thereof under various conditions of stringency (See, for example, Wahl and Berger (1987) Methods Enzymol. 152: 399-407; and Kimmel (1987) Methods Enzymol. 152: 507-511). With regard to hybridization, conditions that are highly stringent, and means for achieving them, are well known in the art. See, for example, Sambrook et al. (1989) “Molecular Cloning: A Laboratory Manual” (2nd ed., Cold Spring Harbor Laboratory); Berger and Kimmel, eds., (1987) “Guide to Molecular Cloning Techniques”, In Methods in Enzymology:152: 467-469; and Anderson and Young (1985) “Quantitative Filter Hybridisation.” In: Hames and Higgins, ed., Nucleic Acid Hybridisation, A Practical Approach. Oxford, IRL Press, 73-111.

Stability of DNA duplexes is affected by such factors as base composition, length, and degree of base pair mismatch. Hybridization conditions may be adjusted to allow DNAs of different sequence relatedness to hybridize. The melting temperature (Tm) is defined as the temperature when 50% of the duplex molecules have dissociated into their constituent single strands. The melting temperature of a perfectly matched duplex, where the hybridization buffer contains formamide as a denaturing agent, may be estimated by the following equation: DNA-DNA: Tm(° C.)=81.5+16.6(log [Na+])+0.41(% G+C)-0.62(% formamide)-500/L (1) DNA-RNA: Tm(° C.)=79.8+18.5(log [Na+])+0.58(% G+C)+0.12(% G+C).sup.2-0.5(% formamide)-820/L (2) RNA-RNA: Tm(C)=79.8+18.5(log [Na+])+0.58(% G+C)+0.12(% G+C).sup.2-0.35(% formamide)-820/L (3), where L is the length of the duplex formed, [Na+] is the molar concentration of the sodium ion in the hybridization or washing solution, and % G+C is the percentage of (guanine+cytosine) bases in the hybrid. For imperfectly matched hybrids, approximately 1° C. is required to reduce the melting temperature for each 1% mismatch.

Hybridization experiments are generally conducted in a buffer of pH between 6.8 to 7.4, although the rate of hybridization is nearly independent of pH at ionic strengths likely to be used in the hybridization buffer (Anderson et al. (1985) supra). In addition, one or more of the following may be used to reduce non-specific hybridization: sonicated salmon sperm DNA or another non-complementary DNA, bovine serum albumin, sodium pyrophosphate, sodium dodecylsulfate (SDS), polyvinyl-pyrrolidone, ficoll and Denhardt's solution. Dextran sulfate and polyethylene glycol 6000 act to exclude DNA from solution, thus raising the effective probe DNA concentration and the hybridization signal within a given unit of time. In some instances, conditions of even greater stringency may be desirable or required to reduce non-specific and/or background hybridization. These conditions may be created with the use of higher temperature, lower ionic strength and higher concentration of a denaturing agent such as formamide.

Stringency conditions can be adjusted to screen for moderately similar fragments such as homologous sequences from distantly related organisms, or to highly similar fragments. The stringency can be adjusted either during the hybridization step or in the post-hybridization washes. Salt concentration, formamide concentration, hybridization temperature and probe lengths are variables that can be used to alter stringency (as described by the formula above). As a general guidelines high stringency is typically performed at Tm-5° C. to Tm-20° C., moderate stringency at Tm-20° C. to Tm-35° C. and low stringency at Tm-35° SC to Tm-50° C. for duplex >150 base pairs. Hybridization may be performed at low to moderate stringency (25-50° C. below T.sub.m), followed by post-hybridization washes at increasing stringencies. Maximum rates of hybridization in solution are determined empirically to occur at Tm-25° C. for DNA-DNA duplex and Tm-15° C. for RNA-DNA duplex. Optionally, the degree of dissociation may be assessed after each wash step to determine the need for subsequent, higher stringency wash steps.

High stringency conditions may be used to select for nucleic acid sequences with high degrees of identity to the disclosed sequences. An example of stringent hybridization conditions obtained in a filter-based method such as a Southern or northern blot for hybridization of complementary nucleic acids that have more than 100 complementary residues is about 5° C. to 20° C. lower than the thermal melting point (Tm) for the specific sequence at a defined ionic strength and pH. Conditions used for hybridization may include about 0.02 M to about 0.15 M sodium chloride, about 0.5% to about 5% casein, about 0.02% SDS or about 0.1% N-laurylsarcosine, about 0.001 M to about 0.03 M sodium citrate, at hybridization temperatures between about 50° C. and about 70° C. In certain embodiments, high stringency conditions are about 0.02 M sodium chloride, about 0.5% casein, about 0.02% SDS, about 0.001 M sodium citrate, at a temperature of about 50° C. Nucleic acid molecules that hybridize under stringent conditions will typically hybridize to a probe based on either the entire DNA molecule or selected portions, e.g., to a unique subsequence, of the DNA.

Stringent salt concentration will ordinarily be less than about 750 mM NaCl and 75 mM trisodium citrate. Increasingly stringent conditions may be obtained with less than about 500 mM NaCl and 50 mM trisodium citrate, to even greater stringency with less than about 250 mM NaCl and 25 mM trisodium citrate. Low stringency hybridization can be obtained in the absence of organic solvent, e.g., formamide, whereas in certain embodiments high stringency hybridization may be obtained in the presence of at least about 35% formamide, and in other embodiments in the presence of at least about 50% formamide. In certain embodiments, stringent temperature conditions will ordinarily include temperatures of at least about 30° C., and in other embodiment at least about 37° C., and in other embodiments at least about 42° C. with formamide present. Varying additional parameters, such as hybridization time, the concentration of detergent, e.g., sodium dodecyl sulfate (SDS) and ionic strength, are well known to those skilled in the art. Various levels of stringency are accomplished by combining these various conditions as needed. In a certain embodiment, hybridization will occur at 30° C. in 750 mM NaCl, 75 mM trisodium citrate, and 1% SDS. In another embodiment, hybridization will occur at 37° C. in 500 mM NaCl, 50 mM trisodium citrate, 1% SDS, 35% formamide. In another embodiment, hybridization will occur at 42C in 250 mM NaCl, 25 mM trisodium citrate, 1% SDS, 50% formamide. Useful variations on these conditions will be readily apparent to those skilled in the art.

The washing steps that follow hybridization may also vary in stringency; the post-hybridization wash steps primarily determine hybridization specificity, with the most critical factors being temperature and the ionic strength of the final wash solution. Wash stringency can be increased by decreasing salt concentration or by increasing temperature. Stringent salt concentration for the wash steps can be less than about 30 mM NaCl and 3 mM trisodium citrate, and in certain embodiments less than about 15 mM NaCl and 1.5 mM trisodium citrate. For example, the wash conditions may be under conditions of 0.1×SSC to 2.0×SSC and 0.1% SDS at 50-65° C., with, for example, two steps of 10-30 min. One example of stringent wash conditions includes about 2.0×SSC, 0.1% SDS at 65° C. and washing twice, each wash step being about 30 min. The temperature for the wash solutions will ordinarily be at least about 25° C., and for greater stringency at least about 42° C. Hybridization stringency may be increased further by using the same conditions as in the hybridization steps, with the wash temperature raised about 3° C. to about 5° C., and stringency may be increased even further by using the same conditions except the wash temperature is raised about 6° C. to about 9° C. For identification of less closely related homolog, wash steps may be performed at a lower temperature, e.g., 50° C.

An example of a low stringency wash step employs a solution and conditions of at least 25° C. in 30 mM NaCl, 3 mM trisodium citrate, and 0.1% SDS over 30 min. Greater stringency may be obtained at 42° C. in 15 mM NaCl, with 1.5 mM trisodium citrate, and 0.1% SDS over 30 min. Even higher stringency wash conditions are obtained at 65° C.-68° C. in a solution of 15 mM NaCl, 1.5 mM trisodium citrate, and 0.1% SDS. Wash procedures will generally employ at least two final wash steps. Additional variations on these conditions will be readily apparent to those skilled in the art.

Stringency conditions can be selected such that an oligonucleotide that is perfectly complementary to the coding oligonucleotide hybridizes to the coding oligonucleotide with at least about a 5-10× higher signal to noise ratio than the ratio for hybridization of the perfectly complementary oligonucleotide to a nucleic acid. It may be desirable to select conditions for a particular assay such that a higher signal to noise ratio, that is, about 15× or more, is obtained. Accordingly, a subject nucleic acid will hybridize to a unique coding oligonucleotide with at least a 2× or greater signal to noise ratio as compared to hybridization of the coding oligonucleotide to a nucleic acid encoding known polypeptide. The particular signal will depend on the label used in the relevant assay, e.g., a fluorescent label, a calorimetric label, a radioactive label, or the like. Labeled hybridization or PCR probes for detecting related polynucleotide sequences may be produced by oligolabeling, nick translation, end-labeling, or PCR amplification using a labeled nucleotide.

The sequence identities can be determined by analysis with a sequence comparison algorithm or by a visual inspection. Protein and/or nucleic acid sequence identities (homologies) can be evaluated using any of the variety of sequence comparison algorithms and programs known in the art.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of nucleic acids and proteins, the BLAST and BLAST 2.2.2. or FASTA version 3.0t78 algorithms and the default parameters discussed below can be used.

A “comparison window”, as used herein, includes reference to a segment of any one of the number of contiguous positions selected from the group consisting of from 20 to 600, usually about 50 to about 200, more usually about 100 to about 150 in which a sequence can be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2: 482, 1981, by the homology alignment algorithm of Needleman & Wunsch, J. Mol. Biol. 48: 443, 1970, by the search for similarity method of Pearson & Lipman, Proc. Natl. Acad. Sci. U.S.A. 85: 2444, 1988, by computerized implementations of these algorithms (FASTDB (Intelligenetics), BLAST (National Center for Biothedical Information), GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., (1999 Suppl.), Current Protocols in Molecular Biology, Greene Publishing Associates and Wiley Interscience, N.Y., 1987)

An example of an algorithm that is suitable for determining percent sequence identity and sequence similarity is the FASTA algorithm, which is described in Pearson, W. R. & Lipman, D. J., Proc. Natl. Acad. Sci. U.S.A. 85: 2444, 1988. See also W. R. Pearson, Methods Enzymol. 266: 227-258, 1996. Exemplary parameters used in a FASTA alignment of DNA sequences to calculate percent identity are optimized, BL50 Matrix 15: −5, k-tuple=2; joining penalty=40, optimization=28; gap penalty −12; gap length penalty=−2; and width=16.

Another example of algorithm that is suitable for determining percent sequence identity and sequence similarity are the BLAST and BLAST 2.0 algorithms, which are described in Altschul et al., Nuc. Acids Res. 25:3389-3402, 1977; and Altschul et al., J. Mol. Biol. 215:403-410, 1990, respectively. BLAST and BLAST 2.0 are used, with the parameters described herein, to determine percent sequence identity for the nucleic acids and proteins of the invention. Software for performing BLAST analyses is publicly available through the National Center for Biotechnology Information (http://www ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. U.S.A. 89:10915, 1989) alignments (B) of 50, expectation (E) of 10, M=5, N=−4, and a comparison of both strands.

The BLAST algorithm also performs a statistical analysis of the similarity between two sequences (see, e.g., Karlin & Altschul, Proc. Natl. Acad. Sci. U.S.A. 90:5873-5787, 1993). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, less than about 0.01, and less than about 0.001.

Another example of a useful algorithm is PILEUP. PILEUP creates a multiple sequence alignment from a group of related sequences using progressive, pairwise alignments to show relationship and percent sequence identity. It also plots a tree or dendogram showing the clustering relationships used to create the alignment. PILEUP uses a simplification of the progressive alignment method of Feng & Doolittle, J. Mol. Evol. 35:351-360, 1987. The method used is similar to the method described by Higgins & Sharp, CABIOS 5:151-153, 1989. The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. Using PILEUP, a reference sequence is compared to other test sequences to determine the percent sequence identity relationship using the following parameters: default gap weight (3.00), default gap length weight (0.10), and weighted end gaps. PILEUP can be obtained from the GCG sequence analysis software package, e.g., version 7.0 (Devereaux et al., Nuc. Acids Res. 12:387-395, 1984.

Another example of an algorithm that is suitable for multiple DNA and amino acid sequence alignments is the CLUSTALW program (Thompson, J. D. et al., Nucl. Acids. Res. 22:4673-4680, 1994). ClustalW performs multiple pairwise comparisons between groups of sequences and assembles them into a multiple alignment based on homology. Gap open and Gap extension penalties were 10 and 0.05 respectively. For amino acid alignments, the BLOSUM algorithm can be used as a protein weight matrix (Henikoff and Henikoff, Proc. Natl. Acad. Sci. U.S.A. 89:10915-10919, 1992).

“Percent identity” in the context of two or more nucleic acids or polypeptide sequences, refers to the percentage of nucleotides or amino acids that two or more sequences or subsequences contain which are the same. A specified percentage of amino acid residues or nucleotides can be referred to such as: 60% identity, 65%, 70%, 75%, 80%, 85%, 90%, 91%, 92%, 93%, 94%, 95%, 96%, 97%, 98%, or 99% or more identity over a specified region, when compared and aligned for maximum correspondence over a comparison window, or designated region as measured using one of the following sequence comparison algorithms or by manual alignment and visual inspection.

“Substantially identical,” in the context of two nucleic acids or polypeptides, refers to two or more sequences or subsequences that have at least of at least 98%, at least 99% or higher nucleotide or amino acid residue identity, when compared and aligned for maximum correspondence, as measured using one of the following sequence comparison algorithms or by visual inspection.

In other aspects, the invention is directed to expression constructs, for example but not limited to plasmids and vectors which comprise nucleic acid sequences of SEQ ID NO: 1-10, complementary sequences thereof, and/or variants thereof. Such expression constructs can be prepared by any suitable method known in the art. Such expression constructs are suitable for viral nucleic acid and/or protein expression and purification.

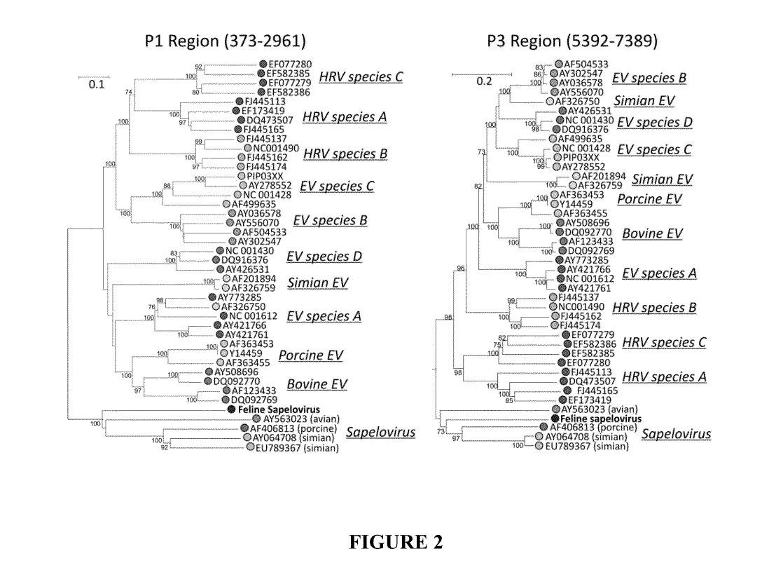

The novel picornavirus shares less than 50% amino acid identity with any know picornavirus reported so far (FIG. 2).

In certain embodiments, protein and/or nucleic acid sequence identities may be evaluated using any of the variety of sequence comparison algorithms and programs known in the art. Such algorithms and programs include, but are by no means limited to, TBLASTN, BLASTP, FASTA, TFASTA, and CLUSTALW (Pearson and Lipman, Proc. Natl. Acad. Sci. USA 85(8):2444-2448, 1988; Altschul et al., J. Mol. Biol. 215(3):403-410, 1990; Thompson et al., Nucleic Acids Res. 22(2):4673-4680, 1994; Higgins et al., Methods Enzymol. 266:383-402, 1996; Altschul et al., J. Mol. Biol. 215(3):403-410, 1990; Altschul et al., Nature Genetics 3:266-272, 1993). In one embodiment, the sequence comparison algorithm is FASTA version 3.0t78 using default parameters.

Isolated Polypeptides

The invention is also directed to isolated polypeptides and variants and derivatives thereof. These polypeptides may be useful for multiple applications, including, but not limited to, generation of antibodies and generation of immunogenic compositions. For example, the invention is directed to an isolated polypeptide having the sequence of any one of SEQ ID NO: 2-18. In certain embodiments, the polypeptides of the present invention can be suitable for use as antigens to detect antibodies against picornavirus represented by SEQ ID NOs: 1, and variants thereof. In other embodiments, the polypeptides of the present invention which comprise antigenic determinants can be used in various immunoassays to identify subjects exposed to and/or samples which comprise picornaviruses represented by SEQ ID NO: 1, and variants thereof.

In one aspect, the invention is directed to polypeptide variants of any one of SEQ ID NO: 2-18. Contemplated variants of any one of SEQ ID NO: 2-18 include but are not limited to polypeptide sequences having at least from about 50% to about 55% identity to that of any one of SEQ ID NO: 2-18. Contemplated variants of any one of SEQ ID NO: 2-18 include but are not limited to polypeptide sequences having at least from about 55.1% to about 60% identity to that of any one of SEQ ID NO: 2-18. Contemplated variants of any one of SEQ ID NO: 2-18 include but are not limited to polypeptide sequences having at least from about 60.1% to about 65% identity to that of any one of SEQ ID NO: 2-18. Contemplated variants of any one of SEQ ID NO: 24-35 include but are not limited to polypeptide sequences having at least from about 65.1% to about 70% identity to that of any one of SEQ ID NO: 2-18. Contemplated variants of any one of SEQ ID NO: 24-35 include but are not limited to polypeptide having at least from about 70.1% to about 75% identity to that of any one of SEQ ID NO: 2-18. Contemplated variants of any one of SEQ ID NO: 24-35 include but are not limited to polypeptide sequences having at least from about 75.1% to about 80% identity to that of any one of SEQ ID NO: 2-18. Contemplated variants of any one of SEQ ID NO: 2-18 include but are not limited to polypeptide sequences having at least from about 80.1% to about 85% identity to that of any one of SEQ ID NO: 2-18. Contemplated variants of any one of SEQ ID NO: 24-35 include but are not limited to polypeptide sequences having at least from about 85.1% to about 90% identity to that of any one of SEQ ID NO: 2-18. Contemplated variant of any one of SEQ ID NO: 2-18 include but are not limited to polypeptide sequences having at least from about 90.1% to about 95% identity to that of any one of SEQ ID NO: 2-18. Contemplated variants of any one of SEQ ID NO: 2-18 include but are not limited to polypeptide sequences having at least from about 95.1% to about 97% identity to that of any one of SEQ ID NO: 2-18. Contemplated variant of any one of SEQ ID NO: 2-18 include but are not limited to polypeptide sequences having at least from about 97.1% to about 99% identity to that of any one of SEQ ID NO: 2-18.

The invention is directed to a polypeptide sequence comprising from about 10 to about 50-consecutive amino acids from any one of SEQ ID NO: 2. The invention is directed to a polypeptide sequence comprising from about 10 to about 100 consecutive amino acids from any one of SEQ ID NO: 2. The invention is directed to a polypeptide sequence comprising from about 10 to about 150 consecutive amino acids from any one of SEQ ID NO: 2. The invention is directed to a polypeptide sequence comprising from about 10 to about 200 consecutive amino acids from any one of SEQ ID NO: 2. The invention is directed to a polypeptide sequence comprising from about 10 to about 250 consecutive amino acids from any one of SEQ ID NO: 2. The invention is directed to a polypeptide sequence comprising from about 10 to about 300 consecutive amino acids from any one of SEQ ID NO: 2. The invention is directed to a polypeptide sequence comprising from about 10 to about 350 consecutive amino acids from any one of SEQ ID NO: 2. The invention is directed to a polypeptide sequence comprising from about 10 to about 400 consecutive amino acids from any one of SEQ ID NO: 2. The invention is directed to a polypeptide sequence comprising from about 10 to about 450 consecutive amino acids from any one of SEQ ID NO: 2. The invention is directed to a polypeptide sequence comprising from about 10 to about 460 consecutive amino acids from any one of SEQ ID NO: 2. The invention is directed to a polypeptide sequence comprising from about 10 to about 470 consecutive amino acids from any one of SEQ ID NO: 2. The invention is directed to a polypeptide sequence comprising from about 10 to about 480 consecutive amino acids from any one of SEQ ID NO: 2. The invention is directed to a polypeptide sequence comprising from about 10 to about 490 consecutive amino acids from any one of SEQ ID NO: 2.

The invention is further directed to polypeptide sequences having from about 50% to about 99% identity to a polypeptide sequence comprising from about 10 to about 490 consecutive amino acids from any one of SEQ ID NO: 2. The invention is further directed to polypeptide sequences having from about 50% to about 99% identity to a polypeptide sequence comprising from about 10 to about 550 consecutive amino acids from any one of SEQ ID NO: 2. The invention is further directed to polypeptide sequences having from about 50% to about 99% identity to a polypeptide sequence comprising from about 10 to about 600 consecutive amino acids from any one of SEQ ID NO: 2. The invention is further directed to polypeptide sequences having from about 50% to about 99% identity to a polypeptide sequence comprising from about 10 to about 650 consecutive amino acids from any one of SEQ ID NO: 2. The invention is further directed to polypeptide sequences having from about 50% to about 99% identity to a polypeptide sequence comprising from about 10 to about 685 consecutive amino acids from any one of SEQ ID NO: 2. In certain embodiments, the invention is directed to isolated and purified peptides.

In a non-limiting example, the invention contemplates modifications to the sequence found in (SEQ ID NO: 1) or the nucleic acid sequence which encode polypeptides of SEQ ID NOs: 3-18, with codons that encode amino acids that are chemically equivalent to the amino acids in the native protein. An amino acid substitution involving the substitution of an amino acid with a chemically equivalent amino acid is known as a conserved amino acid substitution. In a non-limiting example, a conserved amino acid substitution results in a conserved/conservative variant. For example, conservative variants may include, but are not limited to, replacement of an amino acid with one having similar properties (for example, polarity, hydrogen bonding potential, acidic, basic, hydrophobic, aromatic and the like). Amino acid residues with similar properties are well known in the art. For example, the amino acid residues arginine, histidine and lysine are hydrophilic, basic amino acid residues and may therefore be interchangeable. Similar, the amino acid residue isoleucine, which is a hydrophobic amino acid residue, may be replaced with leucine, methionine or valine. Such changes are expected to have little or no effect on the apparent molecular weight or isoelectric point of the polypeptide.

The invention encompasses polypeptides having a lower degree of identity but having sufficient similarity so as to perform one or more of the same functions performed by the polypeptide of the present invention. Similarity is determined by conserved amino acid substitution. Such substitutions are those that substitute a given amino acid in a polypeptide by another amino acid of like characteristics (e.g., chemical properties). According to Cunningham and Wells, Science 244:1081-1085 (1989), such conservative substitutions are likely to be phenotypically silent. Additional guidance concerning which amino acid changes are likely to be phenotypically silent are found in Bowie et al., Science 247:1306-1310 (1990). These publications are incorporated in their entirety by reference to the same extent as if each was specifically and individually disclosed.

Tolerated conservative amino acid substitutions of the present invention involve replacement of the aliphatic or hydrophobic amino acids Ala, Val, Leu and Ile; replacement of the hydroxyl residues Ser and Thr, replacement of the acidic residues Asp and Glu; replacement of the amide residues Asn and Gln, replacement of the basic residues Lys, Arg, and His; replacement of the aromatic residues Phe, Tyr, and Trp, and replacement of the small-sized amino acids Ala, Ser, Thr, Met, and Gly.

In certain embodiments, the present invention also encompasses the conservative substitutions provided in Table 1 below.

TABLE 1

| For Amino | | |
|---|---|---|
| Acid | Code | Replace with any of: |
| Alanine | A | D-Ala, Gly, beta-Ala, L-Cys, D-Cys |
| Arginine | R | D-Arg, Lys, D-Lys, home-Arg, D-homo-Arg, |
| | | Met, Ile, D-Met, D-Ile, Orn, D-Orn |
| Asparagine | N | D-Asn, Asp, D-Asp, Glu, D-Gln, Gln, D-Gln |
| Aspartic Acid | D | D-Asp, D-Asn. Asn, Glu, D-Glu, Gln, D-Gln |
| Cysteine | C | D-Cys, S-Me-Cys, Met, D-Met, Thr, D-Thr |
| Glutamine | Q | D-Gln, Asn, D-Asn, Glu, D-Glu, Asp, D-Asp |
| Glutamic Acid | E | D-Glu, D-Asp, Asp, Asn. D-Asn, Gln, D-Gln |
| Glycine | G | Ala, D-Ala, Pro, D-Pro, β-Ala, Acp |
| Isoleucine | I | D-Ile, Val, D-Val. Leu, D-Leu, Met, D-Met |
| Leucine | L | D-Leu, Val. D-Val, Met, D-Met |
| Lysine | K | D-Lys, Arg, D-Arg, homo-Arg, D-homo-Arg, |
| | | Met, D-Met, Ile, D-Ile, Orn, D-Orn |
| Methionine | M | D-Met, S-Me-Cys, Ile, D-Ile, Leu, D-Leu, Val. |
| | | D-Val |
| Phenylalanine | F | D-Phe, Tyr, D-Thr, L-Dopa, His, D-His, Trp, |
| | | D-Trp, Trans-3,4, or 5-phenylproline, cis-3,4, |
| | | or 5-phenylproline |
| Proline | P | D-Pro, L-1-thioazolidine-4-carboxylic acid, |
| | | D- or L-1-oxazolidine-4-carboxylic acid |
| Serine | S | D-Ser, Thr, D-Thr, allo-Thr, Met, D-Met, |
| | | Met(O), D-Met(O), L-Cys. D-Cys |
| Threonine | T | D-Thr, Ser, D-Ser allo-Thr, Met, D-Met, |
| | | Met(O), D-Met(O), Val, D-Val |
| Tyrosine | Y | D-Tyr, Phe, D-Phe. L-Dopa, His, D-His |
| Valine | V | D-Val, Leu, D-Leu, Ile, D-Ile, Met, D-Met |

Amino acid residues other than those indicated as conserved may also differ in a protein or enzyme so that the percent protein or amino acid sequence similarity (e.g., percent identity or homology) between any two proteins of similar function may vary and may be, for example, from 70% to 99% as determined according to an alignment scheme such as the Cluster Method, wherein similarity is based on the MEGALIGN algorithm. “A conservative variants” of a given polypeptide of the invention also include polypeptides that have at least 60% amino acid sequence identity to the given polypeptide as determined, e.g., by the BLAST or FASTA algorithms.

Antibodies

In another aspect, the invention is directed to an antibody which specifically binds to the feline picorna virus of the invention, or amino acids in the polypeptide of any one of SEQ ID NO: 2-18. In another aspect, the invention is directed to an antibody which specifically binds to amino acids from the polypeptide of any one of SEQ ID NO: 2-18, or their conserved variants, or fragments. In one embodiment the antibody is purified. The antibodies can be polyclonal or monoclonal. The antibodies can also be chimeric (i.e., a combination of sequences from more than one species, for example, a chimeric mouse-human immunoglobulin, mouse-feline), or derived fully from one species, e.g., feline or mouse.

The antibodies of the present invention have various utilities. For example, such antibodies may be used in diagnostic assays to detect the presence or quantification of the polypeptides of the invention in a sample. Such a diagnostic assay may be comprised of at least two steps. The first, subjecting a sample with the antibody, wherein the sample is a tissue (e.g., human, animal, etc.), biological fluid (e.g., blood, urine, sputum, semen, amniotic fluid, saliva, etc.), biological extract (e.g., tissue or cellular homogenate, etc.), a protein microchip (e.g., See Arenkov P, et al., Anal Biochem., 278(2):123-131 (2000)), or a chromatography column, etc. And a second step involving the quantification of antibody bound to the substrate. Alternatively, the method may additionally involve a first step of attaching the antibody, either covalently, electrostatically, or reversibly, to a solid support, and a second step of subjecting the bound antibody to the sample, as defined above and elsewhere herein.

Various diagnostic assay techniques are known in the art, such as competitive binding assays, direct or indirect sandwich assays and immunoprecipitation assays conducted in either heterogeneous or homogenous phases (Zola, Monoclonal Antibodies: A Manual of Techniques, CRC Press, Inc., (1987), pp. 147-158). The antibodies used in the diagnostic assays can be labeled with a detectable moiety. The detectable moiety should be capable of producing, either directly or indirectly, a detectable signal. For example, the detectable moiety may be a radioisotope, such as 2H, ¹⁴C, 32P, or 125I, a florescent or chemiluminescent compound, such as fluorescein isothiocyanate, rhodamine, or luciferin, or an enzyme, such as alkaline phosphatase, beta-galactosidase, green fluorescent protein, or horseradish peroxidase. Any method known in the art for conjugating the antibody to the detectable moiety may be employed, including those methods described by Hunter et al., Nature, 144:945 (1962); Dafvid et al., Biochem., 13:1014 (1974); Pain et al., J. Immunol. Metho., 40:219 (1981); and Nygren, J. Histochem. And Cytochem., 30:407 (1982).

Antibodies directed against the polypeptides and virus of the present invention are useful for the affinity purification of such polypeptides and virus from recombinant cell culture or natural sources. In a non-limiting example, the antibodies against a particular polypeptide are immobilized on a suitable support, such as a SEPHADEX® resin or filter paper, using methods well known in the art. The immobilized antibody then is contacted with a sample containing the polypeptides or virus to be purified, and thereafter the support is washed with a suitable solvent that will remove substantially all the material in the sample except for the desired polypeptides, which are bound to the immobilized antibody. Finally, the support is washed with another suitable solvent that will release the desired polypeptide or virus from the antibody.

In non-limiting embodiments, immunogenic sequences are contained in the capsid proteins VP4, VP2, VP3, and VP1. In order to raise protective antibodies (vaccine) one may use VP1, VP2, VP3, VP4, or any combination thereof. In another embodiment, one can use the whole P1 region (comprised of VP4/VP2/VP3/VP1). The P1 region extends in the full length sequence from aa 65-863 of SEQ ID NO:2, with VP4 from aa 65-114; VP2 aa 115-354; VP3 aa 355-582; VP1 aa 583-863 (see FIG. 1A and SEQ ID NOs: 2, and SEQ ID NO:1). In other embodiments, antibodies can be raised against the entire FeSV. In certain embodiments, the FeSV is inactivated. A skilled artisan can readily determine immunogenic sequences.

Antibodies can bind to other molecules (antigens) via heavy and light chain variable domains, V.sub.H and V.sub.L, respectively. Antibodies include IgG, IgD, IgA, IgM and IgE. The antibodies may be intact immunoglobulin molecules, two full length heavy chains linked by disulfide bonds to two full length light chains, as well as subsequences (i.e. fragments) of immunoglobulin molecules that bind to an epitope of an antigen, or subsequences thereof (i.e. fragments) of immunoglobulin molecules, with or without constant region, that bind to an epitope of an antigen. Antibodies may comprise full length heavy and light chain variable domains, V.sub.H and V.sub.L, individually or in any combination.

The basic immunoglobulin (antibody) structural unit is known to comprise a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V.sub.l) and variable heavy chain (V.sub.H) refer to these light and heavy chains respectively.

Antibodies may exist as intact immunoglobulins or as a number of well characterized fragments produced by digestion with various peptidases. In particular, pepsin digests an antibody below the disulfide linkages in the hinge region to produce F(ab)'.sub.2, a dimer of Fab which itself is a light chain joined to V.sub.H-C.sub.H1 by a disulfide bond. The F(ab)'.sub.2 may be reduced under mild conditions to break the disulfide linkage in the hinge region thereby converting the F(ab)'.sub.2 dimer into an Fab′ monomer. The Fab′ monomer is essentially an Fab with part of the hinge region (see, Fundamental Immunology, W. E. Paul, ed., Raven Press, N.Y. (1993) for more antibody fragment terminology). While the Fab′ domain is defined in terms of the digestion of an intact antibody, one of skill will appreciate that such Fab′ fragments may be synthesized de novo either chemically or by utilizing recombinant DNA methodology.

The Fab′ regions may be derived from antibodies of animal (especially mouse or rat) or human origin or may be chimeric (Morrison et al., Proc Natl. Acad. Sci. USA 81, 6851-6855 (1984) both incorporated by reference herein) or humanized (Jones et al., Nature 321, 522-525 (1986), and published UK patent application No. 8707252, both incorporated by reference herein).

An antibody described in this application can include or be derived from any mammal, such as but not limited to, a human, a mouse, a rabbit, a rat, a dog, a rodent, a primate, or any combination thereof and includes isolated human, primate, rodent, mammalian, chimeric, humanized and/or CDR-grafted or CDR-adapted antibodies, immunoglobulins, cleavage products and other portions and variants thereof.

Antibodies useful in the embodiments of the invention can be derived in several ways well known in the art. In one aspect, the antibodies can be obtained using any of the hybridoma techniques well known in the art, see, e.g., Ausubel, et al., ed., Current Protocols in Molecular Biology, John Wiley & Sons, Inc., NY, N.Y. (1987-2001); Sambrook, et al., Molecular Cloning: A Laboratory Manual, 2.sup.nd Edition, Cold Spring Harbor, N.Y. (1989); Harlow and Lane, Antibodies, A Laboratory Manual, Cold Spring Harbor, N.Y. (1989); Colligan, el al., eds., Current Protocols in Immunology, John Wiley & Sons, Inc., NY (1994-2001); Colligan el al., Current Protocols in Protein Science, John Wiley & Sons, NY, N.Y., (1997-2001). Antibodies properties can be optimized using known methods in the art.

The antibodies may also be obtained from selecting from libraries of such domains or components, e.g. a phage library. A phage library can be created by inserting a library of random oligonucleotides or a library of polynucleotides containing sequences of interest, such as from the B-cells of an immunized animal or human (Smith, G. P. 1985. Science 228: 1315-1317). Antibody phage libraries contain heavy (H) and light (L) chain variable region pairs in one phage allowing the expression of single-chain Fv fragments or Fab fragments (Hoogenboom, et al. 2000, Immunol Today 21(8) 371-8). The diversity of a phagemid library can be manipulated to increase and/or alter the immunospecificities of the monoclonal antibodies of the library to produce and subsequently identify additional, desirable, human monoclonal antibodies. For example, the heavy (H) chain and light (L) chain immunoglobulin molecule encoding genes can be randomly mixed (shuffled) to create new HL pairs in an assembled immunoglobulin molecule. Additionally, either or both the H and L chain encoding genes can be mutagenized in a complementarity determining region (CDR) of the variable region of the immunoglobulin polypeptide, and subsequently screened for desirable affinity and neutralization capabilities. Antibody libraries also can be created synthetically by selecting one or more human framework sequences and introducing collections of CDR cassettes derived from human antibody repertoires or through designed variation (Kretzschmar and von Ruden 2000, Current Opinion in Biotechnology, 13:598-602). The positions of diversity are not limited to CDRs but can also include the framework segments of the variable regions or may include other than antibody variable regions, such as peptides.

Other target binding components which may include other than antibody variable regions are ribosome display, yeast display, and bacterial displays. Ribosome display is a method of translating mRNAs into their cognate proteins while keeping the protein attached to the RNA. The nucleic acid coding sequence is recovered by RT-PCR (Mattheakis, L. C. et al. 1994. Proc Natl Acad Sci USA 91, 9022). Yeast display is based on the construction of fusion proteins of the membrane-associated alpha-agglutinin yeast adhesion receptor, aga1 and aga2, a part of the mating type system (Broder, et al. 1997. Nature Biotechnology, 15:553-7). Bacterial display is based fusion of the target to exported bacterial proteins that associate with the cell membrane or cell wall (Chen and Georgiou 2002. Biotechnol Bioeng, 79:496-503).

In comparison to hybridoma technology, phage and other antibody display methods afford the opportunity to manipulate selection against the antigen target in vitro and without the limitation of the possibility of host effects on the antigen or vice versa.

Specific examples of antibody subsequences include, for example, Fab, Fab′, (Fab′).sub.2, Fv, or single chain antibody (SCA) fragment (e.g., scFv). Subsequences include portions which retain at least part of the function or activity of full length sequence. For example, an antibody subsequence will retain the ability to selectively bind to an antigen even though the binding affinity of the subsequence may be greater or less than the binding affinity of the full length antibody.

Pepsin or papain digestion of whole antibodies can be used to generate antibody fragments. In particular, an Fab fragment consists of a monovalent antigen-binding fragment of an antibody molecule, and can be produced by digestion of a whole antibody molecule with the enzyme papain, to yield a fragment consisting of an intact light chain and a portion of a heavy chain. An (Fab′).sub.2 fragment of an antibody can be obtained by treating a whole antibody molecule with the enzyme pepsin, without subsequent reduction. An Fab′ fragment of an antibody molecule can be obtained from (Fab′).sub.2 by reduction with a thiol reducing agent, which yields a molecule consisting of an intact light chain and a portion of a heavy chain. Two Fab′ fragments are obtained per antibody molecule treated in this manner.

An Fv fragment is a fragment containing the variable region of a light chain V.sub.L and the variable region of a heavy chain V.sub.H expressed as two chains. The association may be non-covalent or may be covalent, such as a chemical cross-linking agent or an intermolecular disulfide bond (Inbar et al., (1972) Proc. Natl. Acad Sci. USA 69:2659; Sandhu (1992) Crit. Rev. Biotech. 12:437).

A single chain antibody (“SCA”) is a genetically engineered or enzymatically digested antibody containing the variable region of a light chain V.sub.L and the variable region of a heavy chain, optionally linked by a flexible linker, such as a polypeptide sequence, in either V.sub.L-linker-V.sub.H orientation or in V.sub.H-linker-V.su.b.L orientation. Alternatively, a single chain Fv fragment can be produced by linking two variable domains via a disulfide linkage between two cysteine residues. Methods for producing scFv antibodies are described, for example, by Whitlow et al., (1991) In: Methods: A Companion to Methods in Enzymology 2:97; U.S. Pat. No. 4,946,778; and Pack et al., (1993) Bio/Technology 11:1271.

Other methods of producing antibody subsequences, such as separation of heavy chains to form monovalent light-heavy chain fragments, further cleavage of fragments, or other enzymatic, chemical, or genetic techniques may also be used, provided that the subsequences bind to the antigen to which the intact antibody binds.

Antibodies used in the invention, include full length antibodies, subsequences (e.g., single chain forms), dimers, trimers, tetramers, pentamers, hexamers or any other higher order oligomer that retains at least a part of antigen binding activity of monomer. Multimers can comprise heteromeric or homomeric combinations of full length antibody, subsequences, unmodified or modified as set forth herein and known in the art. Antibody multimers are useful for increasing antigen avidity in comparison to monomer due to the multimer having multiple antigen binding sites. Antibody multimers are also useful for producing oligomeric (e.g., dimer, trimer, tertamer, etc.) combinations of different antibodies thereby producing compositions of antibodies that are multifunctional (e.g., bifunctional, trifunctional, tetrafunctional, etc.).

Antibodies can be produced through chemical crosslinking of the selected molecules (which have been produced by synthetic means or by expression of nucleic acid that encode the polypeptides) or through recombinant DNA technology combined with in vitro, or cellular expression of the polypeptide, and subsequent oligomerization. Antibodies can be similarly produced through recombinant technology and expression, fusion of hybridomas that produce antibodies with different epitopic specificities, or expression of multiple nucleic acid encoding antibody variable chains with different epitopic specificities in a single cell.

Antibodies may be either joined directly or indirectly through covalent or non-covalent binding, e.g. via a multimerization domain, to produce multimers. A “multimerization domain” mediates non-covalent protein-protein interactions. Specific examples include coiled-coil (e.g., leucine zipper structures) and alpha-helical protein sequences. Sequences that mediate protein-protein binding via Van der Waals' forces, hydrogen bonding or charge-charge bonds are also contemplated as multimerization domains. Additional examples include basic-helix-loop-helix domains and other protein sequences that mediate heteromeric or homomeric protein-protein interactions among nucleic acid binding proteins (e.g., DNA binding transcription factors, such as TAFs).

Antibodies may be directly linked to each other via a chemical cross linking agent or can be connected via a linker sequence (e.g., a peptide sequence) to form multimers.

The antibodies of the present invention can be used to modulate the activity of the polypeptide of any one of SEQ ID NO: 2-18, variants or fragments thereof. In certain aspects, the invention is directed to a method for treating a subject, the method comprising administering to the subject an antibody which specifically binds to amino acids from the polypeptide of any one of SEQ ID NO: 2-18. In certain embodiments, antibody binding to the polypeptide of any one of SEQ ID NO: 2-18 may interfere or inhibit the function of the polypeptide, thus providing a method to inhibit virus propagation and spreading. In certain embodiments, the polypeptide is VP1. In other embodiments, the polypeptide is VP4. Thus the invention provides a method for treating a subject suffering from a disease associated with FeSV.

In other embodiments, the antibodies of the invention can be used to purify polypeptides of any one of SEQ ID NO: 2-18, variants or fragments thereof. In other embodiments, the antibodies of the invention can be used to identify expression and localization of the polypeptide of any one of SEQ ID NO: 2-18, variants, fragments or domains thereof. Analysis of expression and localization of the polypeptide of any one of SEQ ID NO: 2-18 can be useful in determining potential role of the polypeptide of any one of SEQ ID NO: 2-18 in the ethiology and progression of diseases, syndromes and disorders dependent on cellular regulation of iron levels.

In certain aspects, the invention provides therapeutic formulation comprising ready-made antibodies or active fragments thereof for passive immunization against feline pricorna virus.

In other embodiments, the antibodies of the present invention can be used in various immunoassays to identify subjects exposed to and/or samples which comprise antigens from picornaviruses represented by SEQ ID NOs: 1, or variants thereof.

Any suitable immunoassay which can lead to formation of antigen-antibody complex is contemplated by the present invention. Variations and different formats of immunoassays, for example but not limited to ELISA, lateral flow assays for detection of analytes in samples, immunoprecipitation, are known in the art, and are contemplated by the invention. In various embodiments, the antigen and/or the antibody can be labeled by any suitable label or method known in the art, for example but not limited to enzymatic. Immunoassays may use solid supports, or immunoprecipitation. Immunoassays which amplify the signal from the antigen-antibody immune complex are also contemplated.

In certain aspects the invention provides methods for assaying a sample to determine the presence or absence of a picornaviruses comprising SEQ ID NOs: 1, or any fragment thereof, as provided by the invention, and variants thereof. The invention contemplates various methods for assaying a sample, including, but not limited to, methods which can detect the presence of nucleic acids, methods which can detect the presence of antigens, methods which can detect the presence of antibodies against antigens from polypeptides encoded by SEQ ID NO: 1, or polypeptides of SEQ ID NO: 2-18, as provided by the invention, and variants thereof, for example but not limited conserved variants.

Biological samples which can be tested for the presence of FeSV include without limitations cerebrospinal fluid, blood, saliva, throat or nasal swabs or washes, urine, fecal samples or rectal swabs, biopsies, or any combination thereof.

Methods to Grow the Feline Picorna Virus.

Cell lines derived from Feline, canine or primate origin can be used to grow the virus. Samples for virus isolation includes infected tissue or excretory samples, including but not limited to oral or gastrointestinal excreta. Methods to grow and isolate picorna viruses in cell culture are known in the art.

Immunogenic Compositions

In certain aspects, the present invention provides immunogenic compositions capable of inducing an immune response against picornaviruses including the FeSV of the invention comprising SEQ ID NO: 1 or any fragment of SEQ ID NO: 1, a variant of FeSV, any one of the polypeptides of SEQ ID NO: 2-18, any fragment, or any combination thereof. In one embodiment, the immunogenic compositions are capable of ameliorating the symptoms of a picornavirus infection and/or of reducing the duration of a picornavirus infection. In another embodiment, the immunogenic compositions are capable of inducing protective immunity against picornavirus infection. The immunogenic compositions of the invention can be effective against the picornavirus disclosed herein, and may also be cross-reactive with, and effective against, multiple different strains of FeSV, and against other picornaviruses.

The types of immunogenic composition encompassed by the invention include, but are not limited to, attenuated live viral vaccines, inactivated (killed) viral vaccines, including but not limited to whole inactivated virus, and subunit vaccines. The immunogenic compositions and vaccines may contain killed or attenuated feline picorna virus, or virus-like particles, i.e. artificial virus derived from the structural proteins of the virus and lacking the genome, or a feline picorna virus component capable of inducing a protecting immune response. The immunogenic compositions and vaccines may further contain various combinations of the above-mentioned, immunologically active constituents to be administered either simultaneously or at different times. The immunogenic compositions or vaccines may also contain a veterinary or pharmaceutically acceptable carrier. The immunogenic compositions or vaccines described herein can be used for prevention or treatment of feline picorna virus infections or symptoms thereof.

The picornavirus of the invention may be attenuated by removal or disruption of those viral sequences whose products cause or contribute to the disease and symptoms associated with viral infection, and leaving intact those sequences required for viral replication. In this way an attenuated picorna virus can be produced that replicates in subjects, and induces an immune response in subjects, but which does not induce the deleterious disease and symptoms usually associated with viral infection. One of skill in the art can determine which FeSV sequences can or should be removed or disrupted, and which sequences should be left intact, in order to generate an attenuated FeSV suitable for use as a vaccine. Attenuation may be carried out according to the customary methods known in the art.

In non-limiting embodiments, a vaccine for this virus can be made using a FeSv isolate serially passaged/propagated using cell culture (attenuated), an inactivated FeSV virus (non-infective virus) a recombinant virus expressing FeSV structural proteins, a mutated FeSV variant or a artificial FeSV like virus altered in codon usage.

The FeSV of the invention may be also be inactivated, such as by chemical treatment, to “kill” the viruses such that they are no longer capable of replicating or causing disease in subjects, but still induce an immune response in a subject. There are many suitable viral inactivation methods known in the art and one of skill in the art can readily select a suitable method and produce an inactivated “killed” virus suitable for use as a vaccine. As a non-limiting example see US Pub. 20100226938, the contents of which are herein incorporated by reference.

The immunogenic compositions of the invention may comprise subunit vaccines. Subunit vaccines include nucleic acid vaccines such as DNA vaccines, which contain nucleic acids that encode one or more viral proteins or subunits, or portions of those proteins or subunits. When using such vaccines, the nucleic acid is administered to the subject, and the immunogenic proteins or peptides encoded by the nucleic acid are expressed in the subject, such that an immune response against the proteins or peptides is generated in the subject. Subunit vaccines may also be proteinaceous vaccines, which contain the viral proteins or subunits themselves, or portions of those proteins or subunits.

To make the nucleic acid and DNA vaccines of the invention the viral sequences disclosed herein may be incorporated into a plasmid or expression vector containing the nucleic acid that encodes the viral protein or peptide. Any suitable plasmid or expression vector capable of driving expression of the protein or peptide in the subject may be used. Such plasmids and expression vectors should include a suitable promoter for directing transcription of the nucleic acid. The nucleic acid sequence(s) that encodes the immunogenic protein or peptide may also be incorporated into a suitable recombinant virus for administration to the subject. Examples of suitable viruses include, but are not limited to, vaccinia viruses, retroviruses, adenoviruses and adeno-associated viruses. One of skill in the art could readily select a suitable plasmid, expression vector, or recombinant virus for delivery of the FeSV nucleic acid sequences of the invention.

To produce the proteinaceous vaccines of the invention, the FeSV nucleic acid sequences of the invention are delivered to cultured cells:for example by transfecting cultured cells with plasmids or expression vectors containing the viral nucleic acid sequences, or by infecting cultured cells with recombinant viruses containing the viral nucleic acid sequences. The viral proteins or peptides may then be expressed in the cultured cells and purified. The purified proteins can then be incorporated into compositions suitable for administration to subjects. Methods and techniques for expression and purification of recombinant proteins are well known in the art, and any such suitable methods may be used.

Subunit vaccines of the present invention may encode or contain any of the viral proteins or peptides described herein, or any portions, fragments, derivatives or mutants thereof, that are immunogenic in a subject. One of skill in the art can readily test the immunogenicity of the FeSV proteins and peptides described herein, and can select suitable proteins or peptides to use in subunit vaccines.

The immunogenic compositions of the invention comprise at least one FeSV-derived immunogenic component, such as those described above. The compositions may also comprise one or more additives including, but not limited to, one or more pharmaceutically acceptable carriers, buffers, stabilizers, diluents, preservatives, solubilizers, liposomes or immunomodulatory agents. Suitable immunomodulatory agents include, but are not limited to, adjuvants, cytokines, polynucleotide encoding cytokines, and agents that facilitate cellular uptake of the FeSV-derived immunogenic component.

Immunogenic compositions for use in accordance with the present invention thus may be formulated in a conventional manner using one or more physiologically acceptable carriers comprising excipients and auxiliaries which facilitate processing of the active compounds into preparations which can be used to induce an immunogenic response. These immunogenic compositions may be manufactured in a manner that is itself known.

The immunogenic composition of the invention may be in the form of a complex of the protein(s) or other active ingredient of present invention along with protein or peptide antigens. The protein and/or peptide antigen will deliver a stimulatory signal to both B and T lymphocytes. B lymphocytes will respond to antigen through their surface immunoglobulin receptor. T lymphocytes will respond to antigen through the T cell receptor (TCR) following presentation of the antigen by MHC proteins. MHC and structurally related proteins including those encoded by class I and class II MHC genes on host cells will serve to present the peptide antigen(s) to T lymphocytes. The antigen components could also be supplied as purified MHC-peptide complexes alone or with co-stimulatory molecules that can directly signal T cells. Alternatively antibodies able to bind surface immunoglobulin and other molecules on B cells as well as antibodies able to bind the TCR and other molecules on T cells can be combined with the immunogenic composition of the invention.

The immunogenic composition of the invention may be in the form of a liposome in which protein of the present invention is combined, in'addition to other acceptable carriers, with amphipathic agents such as lipids which exist in aggregated form as micelles, insoluble monolayers, liquid crystals, or lamellar layers in aqueous solution. Suitable lipids for liposomal formulation include, without limitation, monoglycerides, diglycerides, sulfatides, lysolecithins, phospholipids, saponin, bile acids, and the like. Preparation of such liposomal formulations is within the level of skill in the art, as disclosed, for example, in U.S. Pat. Nos. 4,235,871; 4,501,728; 4,837,028; and 4,737,323, all of which are incorporated herein by reference.

Other additives that are useful in vaccine formulations are known and will be apparent to those of skill in the art.

An “immunologically effective amount” of the compositions of the invention may be administered to a subject. As used herein, the term “immunologically effective amount” refers to an amount capable of inducing, or enhancing the induction of, the desired immune response in a subject. The desired response may include, inter alia, inducing an antibody or cell-mediated immune response, or both. The desired response may also be induction of an immune response sufficient to ameliorate the symptoms of a viral infection, reduce the duration of a viral infection, and/or provide protective immunity in a subject against subsequent challenge with a virus. An immunologically effective amount may be an amount that induces actual “protection” against viral infection, meaning the prevention of any of the symptoms or conditions resulting from viral infection in subjects. An immunologically effective amount may also be an amount sufficient to delay the onset of symptoms and conditions associated with infection, reduce the degree or rate of infection, reduce in the severity of any disease or symptom resulting from infection, and reduce the viral load of an infected subject.

One of skill in the art can readily determine what is an “immunologically effective amount” of the compositions of the invention without performing any undue experimentation. An effective amount can be determined by conventional means, starting with a low dose of and then increasing the dosage while monitoring the immunological effects. Numerous factors can be taken into consideration when determining an optimal amount to administer, including the size, age, and general condition of the subject, the presence of other drugs in the subject, the virulence of the particular virus against which the subject is being vaccinated, and the like. The actual dosage and immunization schedule is can be chosen after consideration of the results from various animal studies.

The immunologically effective amount of the immunogenic composition may be administered in a single dose, in divided doses, or using a “prime-boost” regimen. The compositions may be administered by any suitable route, including, but not limited to parenteral, intradermal, transdermal, subcutaneous, intramuscular, intravenous, intraperitoneal, intranasal, oral, or intraocular routes, or by a combination of routes. The compositions may also be administered using a “gun” device which fires particles, such as gold particles, onto which compositions of the present invention have been coated, into the skin of a subject. The skilled artisan will be able to formulate the vaccine composition according to the route chosen.

Viral Purification

Methods of purification of inactivated virus are known in the art and may include one or more of, for instance gradient centrifugation, ultracentrifugation, continuous-flow ultracentrifugation and chromatography, such as ion exchange chromatography, size exclusion chromatography, and liquid affinity chromatography. Additional method of purification include ultrafiltration and dialfiltration. See J P Gregersen “Herstellung von Virussimpfstoffen aus Zellkulturen” Chapter 4.2 in Pharmazeutische Biotechnology (eds. O. Kayser and R H Mueller) Wissenschaftliche Verlagsgesellschaft, Stuttgart, 2000. See also, O'Neil et al., “Virus Harvesting and Affinity Based Liquid Chromatography. A Method for Virus Concentration and Purification”, Biotechnology (1993) 11:173-177; Prior et al., “Process Development for Manufacture of Inactivated HIV-1”, Pharmaceutical Technology (1995) 30-52; and Majhdi et al., “Isolation and Characterization of a Coronavirus from Elk Calves with diarrhea” Journal of Clinical Microbiology (1995) 35(11): 2937-2942.

Other examples of purification methods suitable for use in the invention include polyethylene glycol or ammonium sulfate precipitation (see Trepanier et al., “Concentration of human respiratory syncytial virus using ammonium sulfate, polyethylene glycol or hollow fiber ultrafiltration” Journal of Virological Methods (1981) 3(4):201-211; Hagen et al., “Optimization of Poly(ethylene glycol) Precipitation of Hepatitis Virus Used to prepare VAQTA, a Highly Purified Inactivated Vaccine” Biotechnology Progress (1996) 12:406-412; and Carlsson et al., “Purification of Infectious Pancreatic Necrosis Virus by Anion Exchange Chromatography Increases the Specific Infectivity” Journal of Virological Methods (1994) 47:27-36) as well as ultrafiltration and microfiltration (see Pay et al., Developments in Biological Standardization (1985) 60:171-174; Tsurumi et al., “Structure and filtration performances of improved cuprammonium regenerated cellulose hollow fibre (improved BMM hollow fibre) for virus removal” Polymer Journal (1990) 22(12):1085-1100; and Makino et al., “Concentration of live retrovirus with a regenerated cellulose hollow fibre, BMM”, Archives of Virology (1994) 139(1-2):87-96.).

Viruses can be purified using chromatography, such as ion exchange, chromatography. Chromatic purification allows for the production of large volumes of virus containing suspension. The viral product of interest can interact with the chromatic medium by a simple adsorption/desorption mechanism, and large volumes of sample can be processed in a single load. Contaminants which do not have affinity for the adsorbent pass through the column. The virus material can then be eluted in concentrated form.

Anion exchange resins that may be used include DEAE, EMD TMAE. Cation exchange resins may comprise a sulfonic acid-modified surface. Viruses can be purified using ion exchange chromatography comprising a strong anion exchange resin (e.g. EMD TMAE) for the first step and EMD-SO.sub.3 (cation exchange resin) for the second step. A metal-binding affinity chromatography step can optionally be included for further purification. (See, e.g., WO 97/06243).

A resin such as Fractogel™ EMD. Can also be used This synthetic methacrylate based resin has long, linear polymer chains (so-called “tentacles”) covalently attached. This “tentacle chemistry” allows for a large amount of sterically accessible ligands for the binding of biomolecules without any steric hindrance. This resin also has improved pressure stability.

Column-based liquid affinity chromatography is another purification method that can be used invention. One example of a resin for use in purification method is Matrex™ Cellufine™ Sulfate (MCS). MCS consists of a rigid spherical (approx. 45-105 .mu.m diameter) cellulose matrix of 3,000 Dalton exclusion limit (its pore structure excludes macromolecules), with a low concentration of sulfate ester functionality on the 6-position of cellulose. As the functional ligand (sulfate ester) is relatively highly dispersed, it presents insufficient cationic charge density to allow for most soluble proteins to adsorb onto the bead surface. Therefore the bulk of the protein found in typical virus pools (cell culture supernatants, e.g. pyrogens and most contaminating proteins, as well as nucleic acids and endotoxins) are washed from the column and a degree of purification of the bound virus is achieved.

The rigid, high-strength beads of MCS tend to resist compression. The pressure/flow characteristics the MCS resin permit high linear flow rates allowing high-speed processing, even in large columns, making it an easily scalable unit operation. In addition a chromatographic purification step with MCS provides increased assurance of safety and product sterility, avoiding excessive product handling and safety concerns. As endotoxins do not bind to it, the MCS purification step allows a rapid and contaminant free depyrogenation. Gentle binding and elution conditions provide high capacity and product yield. The MCS resin therefore represents a simple, rapid, effective, and cost-saving means for concentration, purification and depyrogenation. In addition, MCS resins can be reused repeatedly.

Inactivated viruses may be further purified by gradient centrifugation, or density gradient centrifugation. For commercial scale operation a continuous flow sucrose gradient centrifugation would be an option. This method is widely used to purify antiviral vaccines and is known to one skilled in the art (See J P Gregersen “Flerstellung von Virussimpfstoffen aus Zellkulturen” Chapter 4.2 in Pharmazeutische Biotechnology (eds. 0. Kayser and R H Mueller)

Wissenschaftliche Verlagsgesellschaft, Stuttgart, 2000.)

Additional purification methods which may be used to purify viruses of the invention include the use of a nucleic acid degrading agent, a nucleic acid degrading enzyme, such as a nuclease having DNase and RNase activity, or an endonuclease, such as from Serratia marcescens, commercially available as Benzonase™, membrane adsorbers with anionic functional groups (e.g. Sartobind™) or additional chromatographic steps with anionic functional groups (e.g. DEAE or TMAE). An ultrafiltration/dialfiltration and final sterile filtration step could also be added to the purification method.

The purified viral preparation of the invention is substantially free of contaminating proteins derived from the cells or cell culture and can comprises less than about 1000, 500, 250, 150, 100, or 50 pg cellular nucleic acid/.mu.g virus antigen, and less than about 1000, 500, 250, 150, 100, or 50 pg cellular nucleic acid/dose. The purified viral preparation can also comprises less than about 20 pg or less than about 10 pg. Methods of measuring host cell nucleic acid levels in a viral sample are known in the art. Standardized methods approved or recommended by regulatory authorities such as the WHO or the FDA can be used.

It will be readily apparent to those skilled in the relevant arts that other suitable modifications and adaptations to the methods and applications described herein can be made without departing from the scope of the invention or any embodiment thereof.

The following examples illustrate the invention described herein, and are set forth to aid in the understanding of the invention, and should not be construed to limit in any way the scope of the invention as defined in the claims which follow thereafter.

The following methods can be used in connection with the embodiments of the invention.

### EXAMPLES

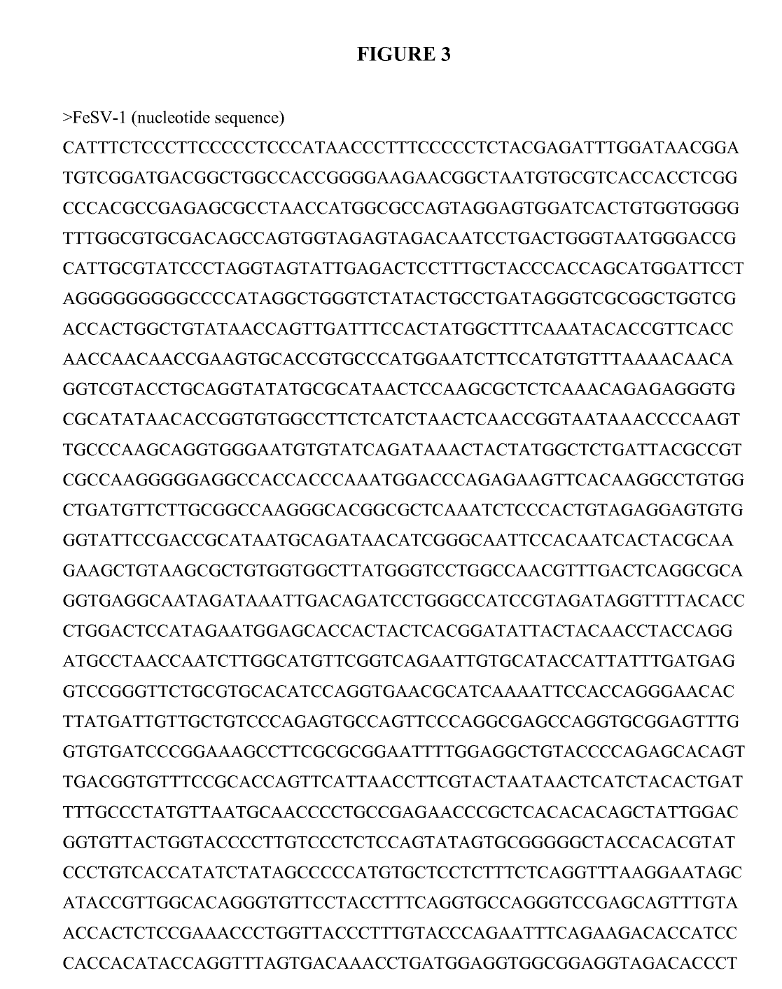

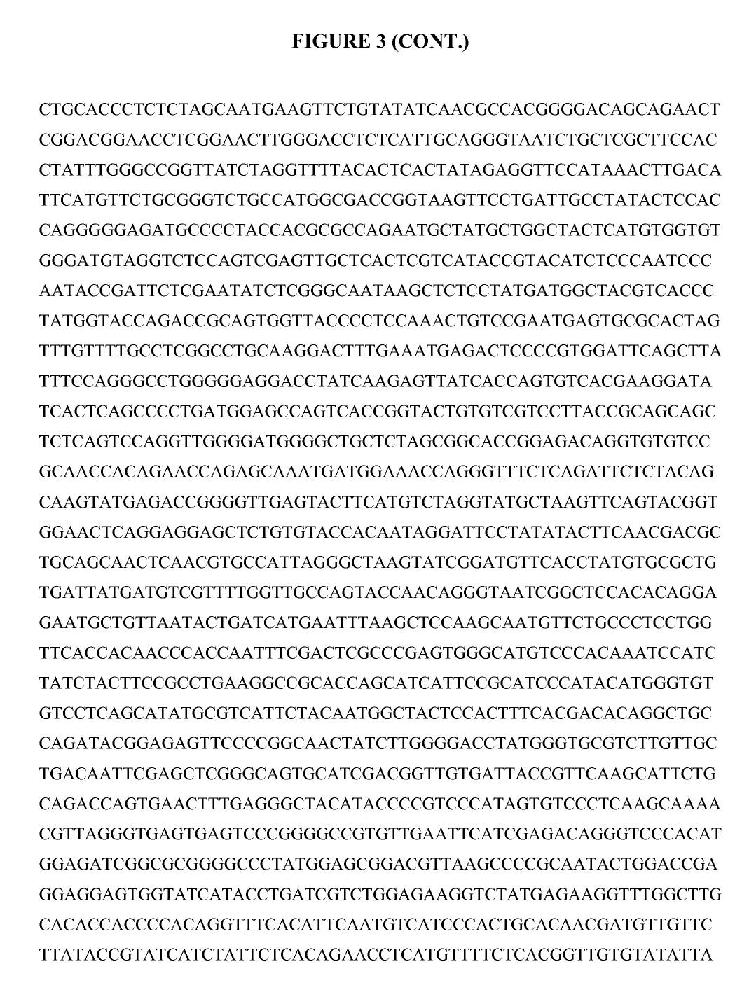

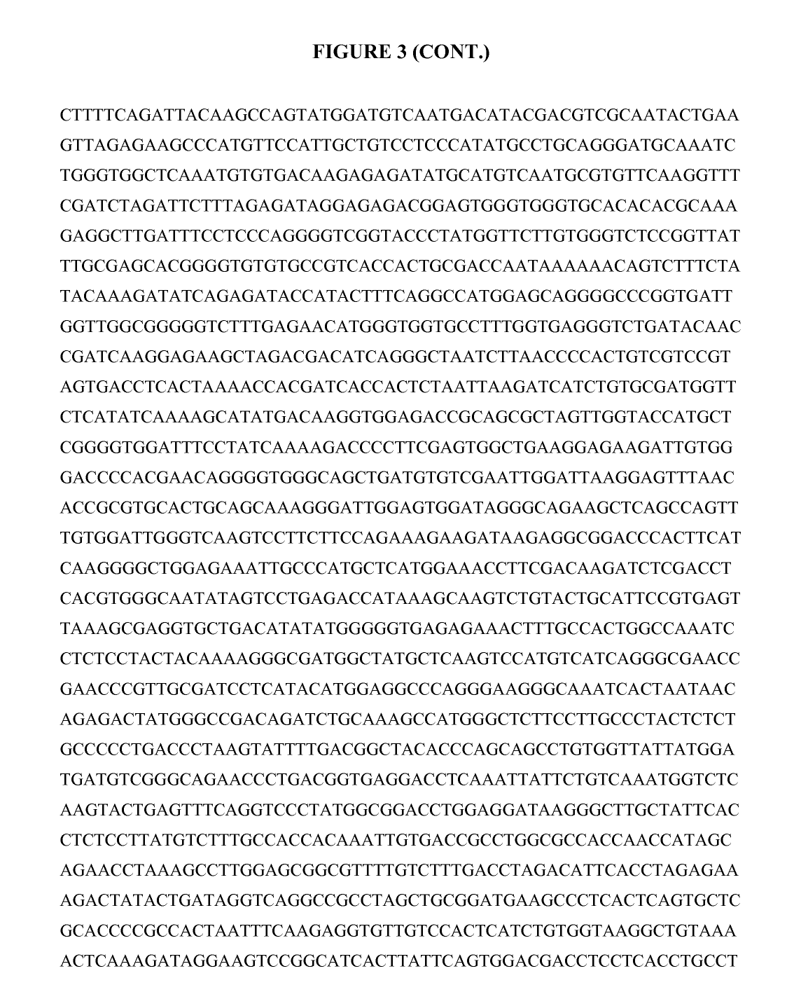

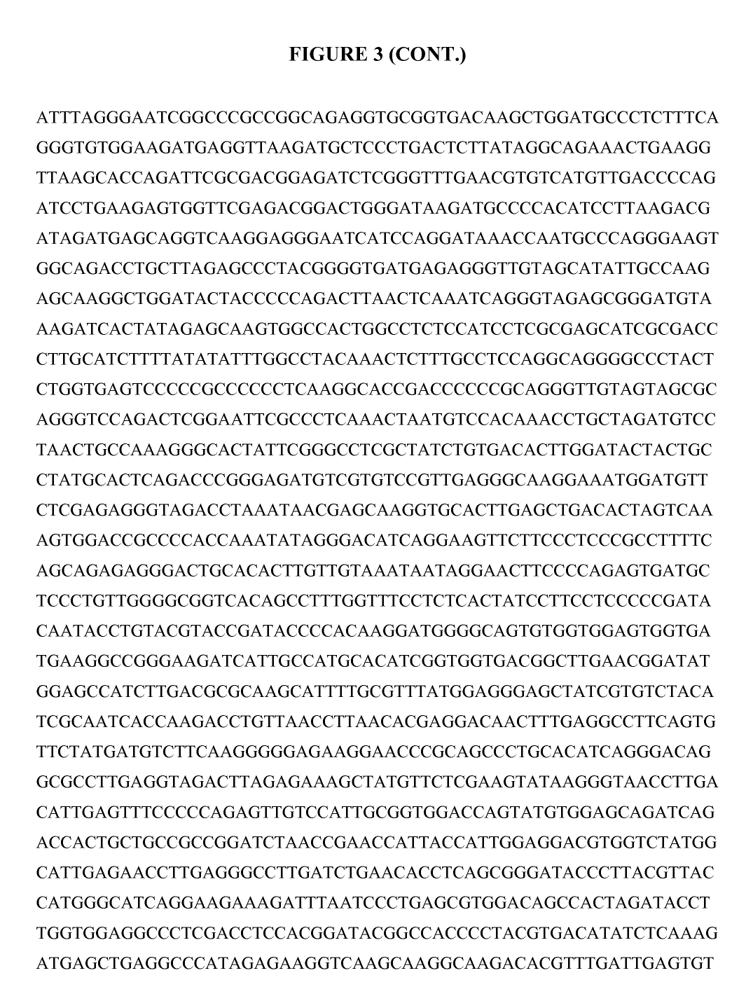

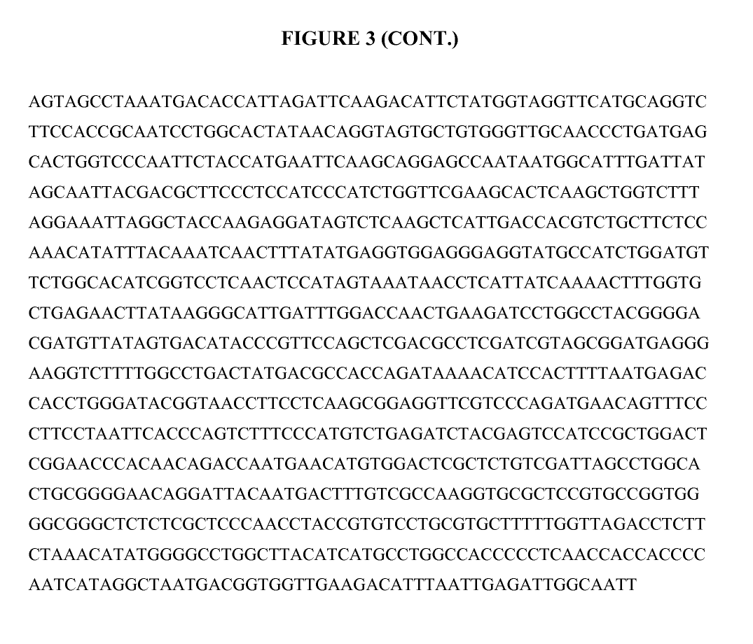

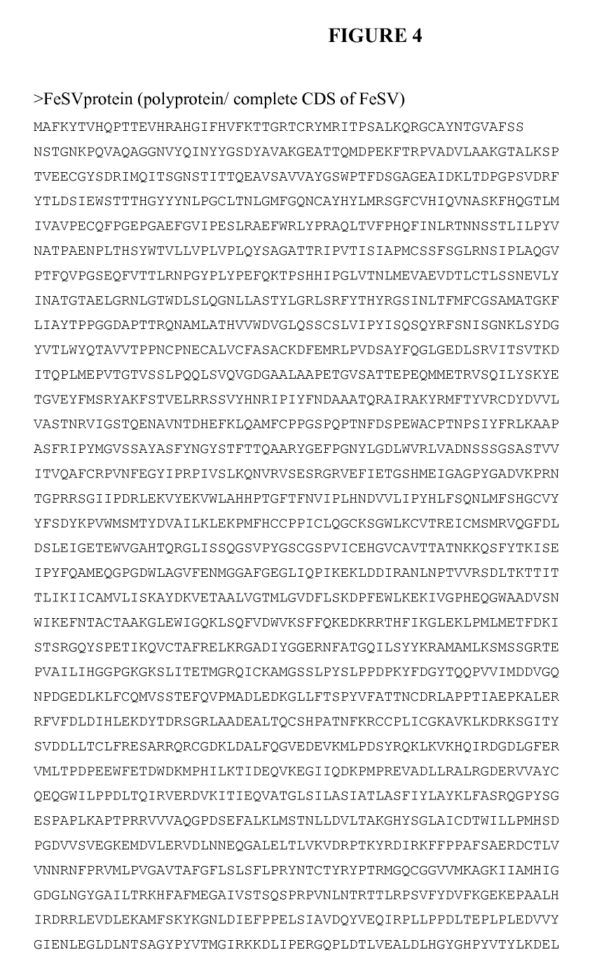

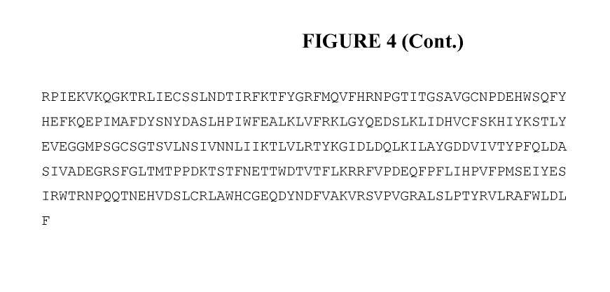

Described herein is a highly divergent picornavirus species found in several cats suffering multiple organ failure and wasting disease. The nucleotide sequence, translated protein sequence of this new virus provisionally named Feline Spelovirus (FeSV) are provided (FIG. 3 and FIG. 4). Phylogenetic analysis based on nucleotide and protein alignments confirms FESV as unique and highly divergent with respect to other known sapeloviruses.

1. An isolated nucleic acid comprising SEQ ID NO: 1.

2. (canceled)

3. An isolated nucleic acid comprising from 10 to 7490 consecutive nucleotides selected from: SEQ ID NO: 1, a sequence complementary to SEQ ID NO: 1, a sequence having about 85% identity to SEQ ID NO: 1, or a sequence having about 85% identity to a sequence complementary to SEQ ID NO: 1, wherein the % identity is determined by analysis with a sequence comparison algorithm.

4. An isolated nucleic acid comprising a nucleic acid encoding any one of the peptide of SEQ ID NOs: 2-18, or a conserved variant of any one of the peptide of SEQ ID NO: 2-18, or a variant thereof.

5. (canceled)

6. (canceled)

7. A replicable vector comprising any one of the nucleic acids of claims 1-4.

8. An isolated peptide comprising any one of the peptides of SEQ ID NOs: 2-18, or a conserved variant of SEQ ID NOs: 2-18.

9. (canceled)

10. An immunogenic composition comprising FeSV, a component of FeSV, or a combination thereof.

11. The immunogenic composition of claim 10, wherein the component is a nucleic acid of FeSV or a fragment thereof, or a peptide of FeSV, or a fragment thereof.

12. The immunogenic composition of claim 11, wherein peptide is P1, VP1, VP2, VP3, VP4, or any combination thereof.

13. The immunogenic composition of claim 10, wherein the FeSV is attenuated, inactivated, or a combination thereof.

14. A pharmaceutical composition for the treatment of a feline picorna virus infection or symptoms thereof, comprising an immunogenic composition comprising FeSV, an immunogenic composition comprising a component of FeSV, an antibody against FeSV, an antibody against a component of FeSV, or a combination thereof.

15. A method to treat, prevent or reduce the severity of a feline picorna virus infection or symptoms thereof, comprising administering a therapeutically effective amount of the pharmaceutical composition of claim 14.

16. An isolated nucleic acid comprising 10 to 30 consecutive nucleotides selected from SEQ ID NO: 1, or a sequence complementary to SEQ ID NO: 1.

17. An isolated nucleic acid comprising 10 to 30 consecutive nucleotides selected from SEQ ID NO: 19, positions 1 to 372 of SEQ ID NO: 1, which is the 5′UTR of SEQ ID NO: 1, SEQ ID NO: 20, positions 2962 to 7490 of SEQ ID NO: 1, SEQ ID NO: 21, positions 6007 to 7389 of SEQ ID NO: 1, or a sequence complementary to SEQ ID NOs: 19, 20, or 21.

18. A kit comprising at least one isolated nucleic acid of claim 16 or 17 and instructions for use.

19. An isolated antibody that specifically binds to FeSV encoded by SEQ ID NO: 1 or a degenerate variant of SEQ ID NO: 1, or binds to a component derived from the FeSV encoded by SEQ ID NO: 1 or a degenerate variant of SEQ ID NO: 1.

20. The antibody of claim 19, wherein the antibody specifically binds to any one of the peptides of SEQ ID NOs: 2-18, or a any combination thereof, or a fragment thereof.

21. An isolated antibody which binds to one or more of P1, (SEQ ID NO: 8), VP1 (SEQ ID NO: 7), VP2, (SEQ ID NO: 5), VP3 (SEQ ID NO: 6), or VP4 (SEQ ID NO: 4) of FeSV.

22. A kit comprising an antibody of claim 20 or 21 and instructions for use.

23. A method to detect FeSV in a biological sample, the method comprising determining the presence or absence in a biological sample from a subject in need thereof of: FeSV, a component of FeSV, an antibody that specifically binds to an epitope comprised in FeSV, or an antibody that specifically binds to an epitope comprised in a component of FeSV or an epitope comprised within any one of SEQ ID NOs: 2-18, or any combination thereof.

24. The method of claim 23, wherein determining is carried out by PCR, immunodetection, immunohistochemistry, in situ hybridization, Nucleic acid sequence based amplification (NASBA) method, by isolating or growing FeSV in cell culture, or any combination thereof.

25. The method of claim 23, wherein the biological sample is from a cat, a dog, or a primate.

26. A method for determining the presence or absence of FeSV in a biological sample, the method comprising:
a) contacting nucleic acid from a biological sample with at least one primer which is a nucleic acid of claim 16 or 17,
b) subjecting the nucleic acid and the primer to amplification conditions, and
c) determining the presence or absence of amplification product, wherein the presence of amplification product indicates the presence of RNA associated with FeSV in the sample.

27. A method for determining the presence or absence of FeSV in a biological sample, the method comprising:
a) contacting a biological sample with an antibody that specifically binds to a FeSV; P1, VP1, VP2, VP3 or VP4 polypeptide encoded by SEQ ID NO:1; or any combination thereof, and
b) determining whether or not the antibody binds to an antigen in the biological sample, wherein binding indicates that the biological sample contains FeSV.

28. The method of claim 27, wherein the determining comprises use of a lateral flow assay or ELISA.

29. The method of claim 27, wherein the determining comprises determining whether the antibodies are IgM antibodies, wherein detection of IgM antibodies is indicative of a recent infection of the sample by a picornavirus FeSV.

30. The method of claim 27, wherein the antibody is any of the antibodies described herein.


SEQUENCE LISTING (21 sequences)

SEQ ID NO: 1 - DNA, 7490 bp, Feline sapelovirus
catttctcccttccccctcccataaccctttccccctctacgagatttggataacggatgtcggatgacggctggccaccggggaagaacggctaatgtgcgtcaccacctcggcccacgccgagagcgcctaaccatggcgccagtaggagtggatcactgtggtggggtttggcgtgcgacagccagtggtagagtagacaatcctgactgggtaatgggaccgcattgcgtatccctaggtagtattgagactcctttgctacccaccagcatggattcctagggggggggccccataggctgggtctatactgcctgatagggtcgcggctggtcgaccactggctgtataaccagttgatttccactatggctttcaaatacaccgttcaccaaccaacaaccgaagtgcaccgtgcccatggaatcttccatgtgtttaaaacaacaggtcgtacctgcaggtatatgcgcataactccaagcgctctcaaacagagagggtgcgcatataacaccggtgtggccttctcatctaactcaaccggtaataaaccccaagttgcccaagcaggtgggaatgtgtatcagataaactactatggctctgattacgccgtcgccaagggggaggccaccacccaaatggacccagagaagttcacaaggcctgtggctgatgttcttgcggccaagggcacggcgctcaaatctcccactgtagaggagtgtgggtattccgaccgcataatgcagataacatcgggcaattccacaatcactacgcaagaagctgtaagcgctgtggtggcttatgggtcctggccaacgtttgactcaggcgcaggtgaggcaatagataaattgacagatcctgggccatccgtagataggttttacaccctggactccatagaatggagcaccactactcacggatattactacaacctaccaggatgcctaaccaatcttggcatgttcggtcagaattgtgcataccattatttgatgaggtccgggttctgcgtgcacatccaggtgaacgcatcaaaattccaccagggaacacttatgattgttgctgtcccagagtgccagttcccaggcgagccaggtgcggagtttggtgtgatcccggaaagccttcgcgcggaattttggaggctgtaccccagagcacagttgacggtgtttccgcaccagttcattaaccttcgtactaataactcatctacactgattttgccctatgttaatgcaacccctgccgagaacccgctcacacacagctattggacggtgttactggtaccccttgtccctctccagtatagtgcgggggctaccacacgtatccctgtcaccatatctatagcccccatgtgctcctctttctcaggtttaaggaatagcataccgttggcacagggtgttcctacctttcaggtgccagggtccgagcagtttgtaaccactctccgaaaccctggttaccctttgtacccagaatttcagaagacaccatcccaccacataccaggtttagtgacaaacctgatggaggtggcggaggtagacaccctctgcaccctctctagcaatgaagttctgtatatcaacgccacggggacagcagaactcggacggaacctcggaacttgggacctctcattgcagggtaatctgctcgcttccacctatttgggccggttatctaggttttacactcactatagaggttccataaacttgacattcatgttctgcgggtctgccatggcgaccggtaagttcctgattgcctatactccaccagggggagatgcccctaccacgcgccagaatgctatgctggctactcatgtggtgtgggatgtaggtctccagtcgagttgctcactcgtcataccgtacatctcccaatcccaataccgattctcgaatatctcgggcaataagctctcctatgatggctacgtcaccctatggtaccagaccgcagtggttacccctccaaactgtccgaatgagtgcgcactagtttgttttgcctcggcctgcaaggactttgaaatgagactccccgtggattcagcttatttccagggcctgggggaggacctatcaagagttatcaccagtgtcacgaaggatatcactcagcccctgatggagccagtcaccggtactgtgtcgtccttaccgcagcagctctcagtccaggttggggatggggctgctctagcggcaccggagacaggtgtgtccgcaaccacagaaccagagcaaatgatggaaaccagggtttctcagattctctacagcaagtatgagaccggggttgagtacttcatgtctaggtatgctaagttcagtacggtggaactcaggaggagctctgtgtaccacaataggattcctatatacttcaacgacgctgcagcaactcaacgtgccattagggctaagtatcggatgttcacctatgtgcgctgtgattatgatgtcgttttggttgccagtaccaacagggtaatcggctccacacaggagaatgctgttaatactgatcatgaatttaagctccaagcaatgttctgccctcctggttcaccacaacccaccaatttcgactcgcccgagtgggcatgtcccacaaatccatctatctacttccgcctgaaggccgcaccagcatcattccgcatcccatacatgggtgtgtcctcagcatatgcgtcattctacaatggctactccactttcacgacacaggctgccagatacggagagttccccggcaactatcttggggacctatgggtgcgtcttgttgctgacaattcgagctcgggcagtgcatcgacggttgtgattaccgttcaagcattctgcagaccagtgaactttgagggctacataccccgtcccatagtgtccctcaagcaaaacgttagggtgagtgagtcccggggccgtgttgaattcatcgagacagggtcccacatggagatcggcgcggggccctatggagcggacgttaagccccgcaatactggaccgaggaggagtggtatcatacctgatcgtctggagaaggtctatgagaaggtttggcttgcacaccaccccacaggtttcacattcaatgtcatcccactgcacaacgatgttgttcttataccgtatcatctattctcacagaacctcatgttttctcacggttgtgtatattacttttcagattacaagccagtatggatgtcaatgacatacgacgtcgcaatactgaagttagagaagcccatgttccattgctgtcctcccatatgcctgcagggatgcaaatctgggtggctcaaatgtgtgacaagagagatatgcatgtcaatgcgtgttcaaggtttcgatctagattctttagagataggagagacggagtgggtgggtgcacacacgcaaagaggcttgatttcctcccaggggtcggtaccctatggttcttgtgggtctccggttatttgcgagcacggggtgtgtgccgtcaccactgcgaccaataaaaaacagtctttctatacaaagatatcagagataccatactttcaggccatggagcaggggcccggtgattggttggcgggggtctttgagaacatgggtggtgcctttggtgagggtctgatacaaccgatcaaggagaagctagacgacatcagggctaatcttaaccccactgtcgtccgtagtgacctcactaaaaccacgatcaccactctaattaagatcatctgtgcgatggttctcatatcaaaagcatatgacaaggtggagaccgcagcgctagttggtaccatgctcggggtggatttcctatcaaaagaccccttcgagtggctgaaggagaagattgtgggaccccacgaacaggggtgggcagctgatgtgtcgaattggattaaggagtttaacaccgcgtgcactgcagcaaagggattggagtggatagggcagaagctcagccagtttgtggattgggtcaagtccttcttccagaaagaagataagaggcggacccacttcatcaaggggctggagaaattgcccatgctcatggaaaccttcgacaagatctcgacctcacgtgggcaatatagtcctgagaccataaagcaagtctgtactgcattccgtgagttaaagcgaggtgctgacatatatgggggtgagagaaactttgccactggccaaatcctctcctactacaaaagggcgatggctatgctcaagtccatgtcatcagggcgaaccgaacccgttgcgatcctcatacatggaggcccagggaagggcaaatcactaataacagagactatgggccgacagatctgcaaagccatgggctcttccttgccctactctctgccccctgaccctaagtattttgacggctacacccagcagcctgtggttattatggatgatgtcgggcagaaccctgacggtgaggacctcaaattattctgtcaaatggtctcaagtactgagtttcaggtccctatggcggacctggaggataagggcttgctattcacctctccttatgtctttgccaccacaaattgtgaccgcctggcgccaccaaccatagcagaacctaaagccttggagcggcgttttgtctttgacctagacattcacctagagaaagactatactgataggtcaggccgcctagctgcggatgaagccctcactcagtgctcgcaccccgccactaatttcaagaggtgttgtccactcatctgtggtaaggctgtaaaactcaaagataggaagtccggcatcacttattcagtggacgacctcctcacctgcctatttagggaatcggcccgccggcagaggtgcggtgacaagctggatgccctctttcagggtgtggaagatgaggttaagatgctccctgactcttataggcagaaactgaaggttaagcaccagattcgcgacggagatctcgggtttgaacgtgtcatgttgaccccagatcctgaagagtggttcgagacggactgggataagatgccccacatccttaagacgatagatgagcaggtcaaggagggaatcatccaggataaaccaatgcccagggaagtggcagacctgcttagagccctacggggtgatgagagggttgtagcatattgccaagagcaaggctggatactacccccagacttaactcaaatcagggtagagcgggatgtaaagatcactatagagcaagtggccactggcctctccatcctcgcgagcatcgcgacccttgcatcttttatatatttggcctacaaactctttgcctccaggcaggggccctactctggtgagtcccccgcccccctcaaggcaccgaccccccgcagggttgtagtagcgcagggtccagactcggaattcgccctcaaactaatgtccacaaacctgctagatgtcctaactgccaaagggcactattcgggcctcgctatctgtgacacttggatactactgcctatgcactcagacccgggagatgtcgtgtccgttgagggcaaggaaatggatgttctcgagagggtagacctaaataacgagcaaggtgcacttgagctgacactagtcaaagtggaccgccccaccaaatatagggacatcaggaagttcttccctcccgccttttcagcagagagggactgcacacttgttgtaaataataggaacttccccagagtgatgctccctgttggggcggtcacagcctttggtttcctctcactatccttcctcccccgatacaatacctgtacgtaccgataccccacaaggatggggcagtgtggtggagtggtgatgaaggccgggaagatcattgccatgcacatcggtggtgacggcttgaacggatatggagccatcttgacgcgcaagcattttgcgtttatggagggagctatcgtgtctacatcgcaatcaccaagacctgttaaccttaacacgaggacaactttgaggccttcagtgttctatgatgtcttcaagggggagaaggaacccgcagccctgcacatcagggacaggcgccttgaggtagacttagagaaagctatgttctcgaagtataagggtaaccttgacattgagtttcccccagagttgtccattgcggtggaccagtatgtggagcagatcagaccactgctgccgccggatctaaccgaaccattaccattggaggacgtggtctatggcattgagaaccttgagggccttgatctgaacacctcagcgggatacccttacgttaccatgggcatcaggaagaaagatttaatccctgagcgtggacagccactagataccttggtggaggccctcgacctccacggatacggccacccctacgtgacatatctcaaagatgagctgaggcccatagagaaggtcaagcaaggcaagacacgtttgattgagtgtagtagcctaaatgacaccattagattcaagacattctatggtaggttcatgcaggtcttccaccgcaatcctggcactataacaggtagtgctgtgggttgcaaccctgatgagcactggtcccaattctaccatgaattcaagcaggagccaataatggcatttgattatagcaattacgacgcttccctccatcccatctggttcgaagcactcaagctggtctttaggaaattaggctaccaagaggatagtctcaagctcattgaccacgtctgcttctccaaacatatttacaaatcaactttatatgaggtggagggaggtatgccatctggatgttctggcacatcggtcctcaactccatagtaaataacctcattatcaaaactttggtgctgagaacttataagggcattgatttggaccaactgaagatcctggcctacggggacgatgttatagtgacatacccgttccagctcgacgcctcgatcgtagcggatgagggaaggtcttttggcctgactatgacgccaccagataaaacatccacttttaatgagaccacctgggatacggtaaccttcctcaagcggaggttcgtcccagatgaacagtttcccttcctaattcacccagtctttcccatgtctgagatctacgagtccatccgctggactcggaacccacaacagaccaatgaacatgtggactcgctctgtcgattagcctggcactgcggggaacaggattacaatgactttgtcgccaaggtgcgctccgtgccggtggggcgggctctctcgctcccaacctaccgtgtcctgcgtgctttttggttagacctcttctaaacatatggggcctggcttacatcatgcctggccaccccctcaaccaccaccccaatcataggctaatgacggtggttgaagacatttaattgagattggcaatt

SEQ ID NO: 2 - protein, 2337 aa, Feline sapelovirus
MAFKYTVHQPTTEVHRAHGIFHVFKTTGRTCRYMRITPSALKQRGCAYNTGVAFSSNSTGNKPQVAQAGGNVYQINYYGSDYAVAKGEATTQMDPEKFTRPVADVLAAKGTALKSPTVEECGYSDRIMQITSGNSTITTQEAVSAVVAYGSWPTFDSGAGEAIDKLTDPGPSVDRFYTLDSIEWSTTTHGYYYNLPGCLTNLGMFGQNCAYHYLMRSGFCVHIQVNASKFHQGTLMIVAVPECQFPGEPGAEFGVIPESLRAEFWRLYPRAQLTVFPHQFINLRTNNSSTLILPYVNATPAENPLTHSYWTVLLVPLVPLQYSAGATTRIPVTISIAPMCSSFSGLRNSIPLAQGVPTFQVPGSEQFVTTLRNPGYPLYPEFQKTPSHHIPGLVTNLMEVAEVDTLCTLSSNEVLYINATGTAELGRNLGTWDLSLQGNLLASTYLGRLSRFYTHYRGSINLTFMFCGSAMATGKFLIAYTPPGGDAPTTRQNAMLATHVVWDVGLQSSCSLVIPYISQSQYRFSNISGNKLSYDGYVTLWYQTAVVTPPNCPNECALVCFASACKDFEMRLPVDSAYFQGLGEDLSRVITSVTKDITQPLMEPVTGTVSSLPQQLSVQVGDGAALAAPETGVSATTEPEQMMETRVSQILYSKYETGVEYFMSRYAKFSTVELRRSSVYHNRIPIYFNDAAATQRAIRAKYRMFTYVRCDYDVVLVASTNRVIGSTQENAVNTDHEFKLQAMFCPPGSPQPTNFDSPEWACPTNPSIYFRLKAAPASFRIPYMGVSSAYASFYNGYSTFTTQAARYGEFPGNYLGDLWVRLVADNSSSGSASTVVITVQAFCRPVNFEGYIPRPIVSLKQNVRVSESRGRVEFIETGSHMEIGAGPYGADVKPRNTGPRRSGIIPDRLEKVYEKVWLAHHPTGFTFNVIPLHNDVVLIPYHLFSQNLMFSHGCVYYFSDYKPVWMSMTYDVAILKLEKPMFHCCPPICLQGCKSGWLKCVTREICMSMRVQGFDLDSLEIGETEWVGAHTQRGLISSQGSVPYGSCGSPVICEHGVCAVTTATNKKQSFYTKISEIPYFQAMEQGPGDWLAGVFENMGGAFGEGLIQPIKEKLDDIRANLNPTVVRSDLTKTTITTLIKIICAMVLISKAYDKVETAALVGTMLGVDFLSKDPFEWLKEKIVGPHEQGWAADVSNWIKEFNTACTAAKGLEWIGQKLSQFVDWVKSFFQKEDKRRTHFIKGLEKLPMLMETFDKISTSRGQYSPETIKQVCTAFRELKRGADIYGGERNFATGQILSYYKRAMAMLKSMSSGRTEPVAILIHGGPGKGKSLITETMGRQICKAMGSSLPYSLPPDPKYFDGYTQQPVVIMDDVGQNPDGEDLKLFCQMVSSTEFQVPMADLEDKGLLFTSPYVFATTNCDRLAPPTIAEPKALERRFVFDLDIHLEKDYTDRSGRLAADEALTQCSHPATNFKRCCPLICGKAVKLKDRKSGITYSVDDLLTCLFRESARRQRCGDKLDALFQGVEDEVKMLPDSYRQKLKVKHQIRDGDLGFERVMLTPDPEEWFETDWDKMPHILKTIDEQVKEGIIQDKPMPREVADLLRALRGDERVVAYCQEQGWILPPDLTQIRVERDVKITIEQVATGLSILASIATLASFIYLAYKLFASRQGPYSGESPAPLKAPTPRRVVVAQGPDSEFALKLMSTNLLDVLTAKGHYSGLAICDTWILLPMHSDPGDVVSVEGKEMDVLERVDLNNEQGALELTLVKVDRPTKYRDIRKFFPPAFSAERDCTLVVNNRNFPRVMLPVGAVTAFGFLSLSFLPRYNTCTYRYPTRMGQCGGVVMKAGKIIAMHIGGDGLNGYGAILTRKHFAFMEGAIVSTSQSPRPVNLNTRTTLRPSVFYDVFKGEKEPAALHIRDRRLEVDLEKAMFSKYKGNLDIEFPPELSIAVDQYVEQIRPLLPPDLTEPLPLEDVVYGIENLEGLDLNTSAGYPYVTMGIRKKDLIPERGQPLDTLVEALDLHGYGHPYVTYLKDELRPIEKVKQGKTRLIECSSLNDTIRFKTFYGRFMQVFHRNPGTITGSAVGCNPDEHWSQFYHEFKQEPIMAFDYSNYDASLHPIWFEALKLVFRKLGYQEDSLKLIDHVCFSKHIYKSTLYEVEGGMPSGCSGTSVLNSIVNNLIIKTLVLRTYKGIDLDQLKILAYGDDVIVTYPFQLDASIVADEGRSFGLTMTPPDKTSTFNETTWDTVTFLKRRFVPDEQFPFLIHPVFPMSEIYESIRWTRNPQQTNEHVDSLCRLAWHCGEQDYNDFVAKVRSVPVGRALSLPTYRVLRAFWLDLF

SEQ ID NO: 3 - protein, 64 aa, Feline sapelovirus
MAFKYTVHQPTTEVHRAHGIFHVFKTTGRTCRYMRITPSALKQRGCAYNTGVAFSSNSTGNKPQ

SEQ ID NO: 4 - protein, 50 aa, Feline sapelovirus
VAQAGGNVYQINYYGSDYAVAKGEATTQMDPEKFTRPVADVLAAKGTALK

SEQ ID NO: 5 - protein, 240 aa, Feline sapelovirus
SPTVEECGYSDRIMQITSGNSTITTQEAVSAVVAYGSWPTFDSGAGEAIDKLTDPGPSVDRFYTLDSIEWSTTTHGYYYNLPGCLTNLGMFGQNCAYHYLMRSGFCVHIQVNASKFHQGTLMIVAVPECQFPGEPGAEFGVIPESLRAEFWRLYPRAQLTVFPHQFINLRTNNSSTLILPYVNATPAENPLTHSYWTVLLVPLVPLQYSAGATTRIPVTISIAPMCSSFSGLRNSIPLAQ

SEQ ID NO: 6 - protein, 228 aa, Feline sapelovirus
GVPTFQVPGSEQFVTTLRNPGYPLYPEFQKTPSHHIPGLVTNLMEVAEVDTLCTLSSNEVLYINATGTAELGRNLGTWDLSLQGNLLASTYLGRLSRFYTHYRGSINLTFMFCGSAMATGKFLIAYTPPGGDAPTTRQNAMLATHVVWDVGLQSSCSLVIPYISQSQYRFSNISGNKLSYDGYVTLWYQTAVVTPPNCPNECALVCFASACKDFEMRLPVDSAYFQGL

SEQ ID NO: 7 - protein, 281 aa, Feline sapelovirus
GEDLSRVITSVTKDITQPLMEPVTGTVSSLPQQLSVQVGDGAALAAPETGVSATTEPEQMMETRVSQILYSKYETGVEYFMSRYAKFSTVELRRSSVYHNRIPIYFNDAAATQRAIRAKYRMFTYVRCDYDVVLVASTNRVIGSTQENAVNTDHEFKLQAMFCPPGSPQPTNFDSPEWACPTNPSIYFRLKAAPASFRIPYMGVSSAYASFYNGYSTFTTQAARYGEFPGNYLGDLWVRLVADNSSSGSASTVVITVQAFCRPVNFEGYIPRPIVSLKQNV

SEQ ID NO: 8 - protein, 799 aa, Feline sapelovirus
VAQAGGNVYQINYYGSDYAVAKGEATTQMDPEKFTRPVADVLAAKGTALKSPTVEECGYSDRIMQITSGNSTITTQEAVSAVVAYGSWPTFDSGAGEAIDKLTDPGPSVDRFYTLDSIEWSTTTHGYYYNLPGCLTNLGMFGQNCAYHYLMRSGFCVHIQVNASKFHQGTLMIVAVPECQFPGEPGAEFGVIPESLRAEFWRLYPRAQLTVFPHQFINLRTNNSSTLILPYVNATPAENPLTHSYWTVLLVPLVPLQYSAGATTRIPVTISIAPMCSSFSGLRNSIPLAQGVPTFQVPGSEQFVTTLRNPGYPLYPEFQKTPSHHIPGLVTNLMEVAEVDTLCTLSSNEVLYINATGTAELGRNLGTWDLSLQGNLLASTYLGRLSRFYTHYRGSINLTFMFCGSAMATGKFLIAYTPPGGDAPTTRQNAMLATHVVWDVGLQSSCSLVIPYISQSQYRFSNISGNKLSYDGYVTLWYQTAVVTPPNCPNECALVCFASACKDFEMRLPVDSAYFQGLGEDLSRVITSVTKDITQPLMEPVTGTVSSLPQQLSVQVGDGAALAAPETGVSATTEPEQMMETRVSQILYSKYETGVEYFMSRYAKFSTVELRRSSVYHNRIPIYFNDAAATQRAIRAKYRMFTYVRCDYDVVLVASTNRVIGSTQENAVNTDHEFKLQAMFCPPGSPQPTNFDSPEWACPTNPSIYFRLKAAPASFRIPYMGVSSAYASFYNGYSTFTTQAARYGEFPGNYLGDLWVRLVADNSSSGSASTVVITVQAFCRPVNFEGYIPRPIVSLKQNV

SEQ ID NO: 9 - protein, 290 aa, Feline sapelovirus
VAQAGGNVYQINYYGSDYAVAKGEATTQMDPEKFTRPVADVLAAKGTALKSPTVEECGYSDRIMQITSGNSTITTQEAVSAVVAYGSWPTFDSGAGEAIDKLTDPGPSVDRFYTLDSIEWSTTTHGYYYNLPGCLTNLGMFGQNCAYHYLMRSGFCVHIQVNASKFHQGTLMIVAVPECQFPGEPGAEFGVIPESLRAEFWRLYPRAQLTVFPHQFINLRTNNSSTLILPYVNATPAENPLTHSYWTVLLVPLVPLQYSAGATTRIPVTISIAPMCSSFSGLRNSIPLAQ

SEQ ID NO: 10 - protein, 517 aa, Feline sapelovirus
AQAGGNVYQINYYGSDYAVAKGEATTQMDPEKFTRPVADVLAAKGTALKSPTVEECGYSDRIMQITSGNSTITTQEAVSAVVAYGSWPTFDSGAGEAIDKLTDPGPSVDRFYTLDSIEWSTTTHGYYYNLPGCLTNLGMFGQNCAYHYLMRSGFCVHIQVNASKFHQGTLMIVAVPECQFPGEPGAEFGVIPESLRAEFWRLYPRAQLTVFPHQFINLRTNNSSTLILPYVNATPAENPLTHSYWTVLLVPLVPLQYSAGATTRIPVTISIAPMCSSFSGLRNSIPLAQGVPTFQVPGSEQFVTTLRNPGYPLYPEFQKTPSHHIPGLVTNLMEVAEVDTLCTLSSNEVLYINATGTAELGRNLGTWDLSLQGNLLASTYLGRLSRFYTHYRGSINLTFMFCGSAMATGKFLIAYTPPGGDAPTTRQNAMLATHVVWDVGLQSSCSLVIPYISQSQYRFSNISGNKLSYDGYVTLWYQTAVVTPPNCPNECALVCFASACKDFEMRLPVDSAYFQGL

SEQ ID NO: 11 - protein, 509 aa, Feline sapelovirus
GVPTFQVPGSEQFVTTLRNPGYPLYPEFQKTPSHHIPGLVTNLMEVAEVDTLCTLSSNEVLYINATGTAELGRNLGTWDLSLQGNLLASTYLGRLSRFYTHYRGSINLTFMFCGSAMATGKFLIAYTPPGGDAPTTRQNAMLATHVVWDVGLQSSCSLVIPYISQSQYRFSNISGNKLSYDGYVTLWYQTAVVTPPNCPNECALVCFASACKDFEMRLPVDSAYFQGLGEDLSRVITSVTKDITQPLMEPVTGTVSSLPQQLSVQVGDGAALAAPETGVSATTEPEQMMETRVSQILYSKYETGVEYFMSRYAKFSTVELRRSSVYHNRIPIYFNDAAATQRAIRAKYRMFTYVRCDYDVVLVASTNRVIGSTQENAVNTDHEFKLQAMFCPPGSPQPTNFDSPEWACPTNPSIYFRLKAAPASFRIPYMGVSSAYASFYNGYSTFTTQAARYGEFPGNYLGDLWVRLVADNSSSGSASTVVITVQAFCRPVNFEGYIPRPIVSLKQNV

SEQ ID NO: 12 - protein, 224 aa, Feline sapelovirus
RVSESRGRVEFIETGSHMEIGAGPYGADVKPRNTGPRRSGIIPDRLEKVYEKVWLAHHPTGFTFNVIPLHNDVVLIPYHLFSQNLMFSHGCVYYFSDYKPVWMSMTYDVAILKLEKPMFHCCPPICLQGCKSGWLKCVTREICMSMRVQGFDLDSLEIGETEWVGAHTQRGLISSQGSVPYGSCGSPVICEHGVCAVTTATNKKQSFYTKISEIPYFQAMEQGP

SEQ ID NO: 13 - protein, 103 aa, Feline sapelovirus
GDWLAGVFENMGGAFGEGLIQPIKEKLDDIRANLNPTVVRSDLTKTTITTLIKIICAMVLISKAYDKVETAALVGTMLGVDFLSKDPFEWLKEKIVGPHEQGW

SEQ ID NO: 14 - protein, 336 aa, Feline sapelovirus
AADVSNWIKEFNTACTAAKGLEWIGQKLSQFVDWVKSFFQKEDKRRTHFIKGLEKLPMLMETFDKISTSRGQYSPETIKQVCTAFRELKRGADIYGGERNFATGQILSYYKRAMAMLKSMSSGRTEPVAILIHGGPGKGKSLITETMGRQICKAMGSSLPYSLPPDPKYFDGYTQQPVVIMDDVGQNPDGEDLKLFCQMVSSTEFQVPMADLEDKGLLFTSPYVFATTNCDRLAPPTIAEPKALERRFVFDLDIHLEKDYTDRSGRLAADEALTQCSHPATNFKRCCPLICGKAVKLKDRKSGITYSVDDLLTCLFRESARRQRCGDKLDALFQGV

SEQ ID NO: 15 - protein, 147 aa, Feline sapelovirus
EDEVKMLPDSYRQKLKVKHQIRDGDLGFERVMLTPDPEEWFETDWDKMPHILKTIDEQVKEGIIQDKPMPREVADLLRALRGDERVVAYCQEQGWILPPDLTQIRVERDVKITIEQVATGLSILASIATLASFIYLAYKLFASRQGP

SEQ ID NO: 16 - protein, 23 aa, Feline sapelovirus
YSGESPAPLKAPTPRRVVVAQGP

SEQ ID NO: 17 - protein, 182 aa, Feline sapelovirus
DSEFALKLMSTNLLDVLTAKGHYSGLAICDTWILLPMHSDPGDVVSVEGKEMDVLERVDLNNEQGALELTLVKVDRPTKYRDIRKFFPPAFSAERDCTLVVNNRNFPRVMLPVGAVTAFGFLSLSFLPRYNTCTYRYPTRMGQCGGVVMKAGKIIAMHIGGDGLNGYGAILTRKHFAFMEGA

SEQ ID NO: 18 - protein, 459 aa, Feline sapelovirus
IVSTSQSPRPVNLNTRTTLRPSVFYDVFKGEKEPAALHIRDRRLEVDLEKAMFSKYKGNLDIEFPPELSIAVDQYVEQIRPLLPPDLTEPLPLEDVVYGIENLEGLDLNTSAGYPYVTMGIRKKDLIPERGQPLDTLVEALDLHGYGHPYVTYLKDELRPIEKVKQGKTRLIECSSLNDTIRFKTFYGRFMQVFHRNPGTITGSAVGCNPDEHWSQFYHEFKQEPIMAFDYSNYDASLHPIWFEALKLVFRKLGYQEDSLKLIDHVCFSKHIYKSTLYEVEGGMPSGCSGTSVLNSIVNNLIIKTLVLRTYKGIDLDQLKILAYGDDVIVTYPFQLDASIVADEGRSFGLTMTPPDKTSTFNETTWDTVTFLKRRFVPDEQFPFLIHPVFPMSEIYESIRWTRNPQQTNEHVDSLCRLAWHCGEQDYNDFVAKVRSVPVGRALSLPTYRVLRAFWLDLF

SEQ ID NO: 19 - DNA, 372 bp, Feline sapelovirus
catttctcccttccccctcccataaccctttccccctctacgagatttggataacggatgtcggatgacggctggccaccggggaagaacggctaatgtgcgtcaccacctcggcccacgccgagagcgcctaaccatggcgccagtaggagtggatcactgtggtggggtttggcgtgcgacagccagtggtagagtagacaatcctgactgggtaatgggaccgcattgcgtatccctaggtagtattgagactcctttgctacccaccagcatggattcctagggggggggccccataggctgggtctatactgcctgatagggtcgcggctggtcgaccactggctgtataaccagttgatttccact

SEQ ID NO: 20 - DNA, 4529 bp, Feline sapelovirus
agggtgagtgagtcccggggccgtgttgaattcatcgagacagggtcccacatggagatcggcgcggggccctatggagcggacgttaagccccgcaatactggaccgaggaggagtggtatcatacctgatcgtctggagaaggtctatgagaaggtttggcttgcacaccaccccacaggtttcacattcaatgtcatcccactgcacaacgatgttgttcttataccgtatcatctattctcacagaacctcatgttttctcacggttgtgtatattacttttcagattacaagccagtatggatgtcaatgacatacgacgtcgcaatactgaagttagagaagcccatgttccattgctgtcctcccatatgcctgcagggatgcaaatctgggtggctcaaatgtgtgacaagagagatatgcatgtcaatgcgtgttcaaggtttcgatctagattctttagagataggagagacggagtgggtgggtgcacacacgcaaagaggcttgatttcctcccaggggtcggtaccctatggttcttgtgggtctccggttatttgcgagcacggggtgtgtgccgtcaccactgcgaccaataaaaaacagtctttctatacaaagatatcagagataccatactttcaggccatggagcaggggcccggtgattggttggcgggggtctttgagaacatgggtggtgcctttggtgagggtctgatacaaccgatcaaggagaagctagacgacatcagggctaatcttaaccccactgtcgtccgtagtgacctcactaaaaccacgatcaccactctaattaagatcatctgtgcgatggttctcatatcaaaagcatatgacaaggtggagaccgcagcgctagttggtaccatgctcggggtggatttcctatcaaaagaccccttcgagtggctgaaggagaagattgtgggaccccacgaacaggggtgggcagctgatgtgtcgaattggattaaggagtttaacaccgcgtgcactgcagcaaagggattggagtggatagggcagaagctcagccagtttgtggattgggtcaagtccttcttccagaaagaagataagaggcggacccacttcatcaaggggctggagaaattgcccatgctcatggaaaccttcgacaagatctcgacctcacgtgggcaatatagtcctgagaccataaagcaagtctgtactgcattccgtgagttaaagcgaggtgctgacatatatgggggtgagagaaactttgccactggccaaatcctctcctactacaaaagggcgatggctatgctcaagtccatgtcatcagggcgaaccgaacccgttgcgatcctcatacatggaggcccagggaagggcaaatcactaataacagagactatgggccgacagatctgcaaagccatgggctcttccttgccctactctctgccccctgaccctaagtattttgacggctacacccagcagcctgtggttattatggatgatgtcgggcagaaccctgacggtgaggacctcaaattattctgtcaaatggtctcaagtactgagtttcaggtccctatggcggacctggaggataagggcttgctattcacctctccttatgtctttgccaccacaaattgtgaccgcctggcgccaccaaccatagcagaacctaaagccttggagcggcgttttgtctttgacctagacattcacctagagaaagactatactgataggtcaggccgcctagctgcggatgaagccctcactcagtgctcgcaccccgccactaatttcaagaggtgttgtccactcatctgtggtaaggctgtaaaactcaaagataggaagtccggcatcacttattcagtggacgacctcctcacctgcctatttagggaatcggcccgccggcagaggtgcggtgacaagctggatgccctctttcagggtgtggaagatgaggttaagatgctccctgactcttataggcagaaactgaaggttaagcaccagattcgcgacggagatctcgggtttgaacgtgtcatgttgaccccagatcctgaagagtggttcgagacggactgggataagatgccccacatccttaagacgatagatgagcaggtcaaggagggaatcatccaggataaaccaatgcccagggaagtggcagacctgcttagagccctacggggtgatgagagggttgtagcatattgccaagagcaaggctggatactacccccagacttaactcaaatcagggtagagcgggatgtaaagatcactatagagcaagtggccactggcctctccatcctcgcgagcatcgcgacccttgcatcttttatatatttggcctacaaactctttgcctccaggcaggggccctactctggtgagtcccccgcccccctcaaggcaccgaccccccgcagggttgtagtagcgcagggtccagactcggaattcgccctcaaactaatgtccacaaacctgctagatgtcctaactgccaaagggcactattcgggcctcgctatctgtgacacttggatactactgcctatgcactcagacccgggagatgtcgtgtccgttgagggcaaggaaatggatgttctcgagagggtagacctaaataacgagcaaggtgcacttgagctgacactagtcaaagtggaccgccccaccaaatatagggacatcaggaagttcttccctcccgccttttcagcagagagggactgcacacttgttgtaaataataggaacttccccagagtgatgctccctgttggggcggtcacagcctttggtttcctctcactatccttcctcccccgatacaatacctgtacgtaccgataccccacaaggatggggcagtgtggtggagtggtgatgaaggccgggaagatcattgccatgcacatcggtggtgacggcttgaacggatatggagccatcttgacgcgcaagcattttgcgtttatggagggagctatcgtgtctacatcgcaatcaccaagacctgttaaccttaacacgaggacaactttgaggccttcagtgttctatgatgtcttcaagggggagaaggaacccgcagccctgcacatcagggacaggcgccttgaggtagacttagagaaagctatgttctcgaagtataagggtaaccttgacattgagtttcccccagagttgtccattgcggtggaccagtatgtggagcagatcagaccactgctgccgccggatctaaccgaaccattaccattggaggacgtggtctatggcattgagaaccttgagggccttgatctgaacacctcagcgggatacccttacgttaccatgggcatcaggaagaaagatttaatccctgagcgtggacagccactagataccttggtggaggccctcgacctccacggatacggccacccctacgtgacatatctcaaagatgagctgaggcccatagagaaggtcaagcaaggcaagacacgtttgattgagtgtagtagcctaaatgacaccattagattcaagacattctatggtaggttcatgcaggtcttccaccgcaatcctggcactataacaggtagtgctgtgggttgcaaccctgatgagcactggtcccaattctaccatgaattcaagcaggagccaataatggcatttgattatagcaattacgacgcttccctccatcccatctggttcgaagcactcaagctggtctttaggaaattaggctaccaagaggatagtctcaagctcattgaccacgtctgcttctccaaacatatttacaaatcaactttatatgaggtggagggaggtatgccatctggatgttctggcacatcggtcctcaactccatagtaaataacctcattatcaaaactttggtgctgagaacttataagggcattgatttggaccaactgaagatcctggcctacggggacgatgttatagtgacatacccgttccagctcgacgcctcgatcgtagcggatgagggaaggtcttttggcctgactatgacgccaccagataaaacatccacttttaatgagaccacctgggatacggtaaccttcctcaagcggaggttcgtcccagatgaacagtttcccttcctaattcacccagtctttcccatgtctgagatctacgagtccatccgctggactcggaacccacaacagaccaatgaacatgtggactcgctctgtcgattagcctggcactgcggggaacaggattacaatgactttgtcgccaaggtgcgctccgtgccggtggggcgggctctctcgctcccaacctaccgtgtcctgcgtgctttttggttagacctcttctaaacatatggggcctggcttacatcatgcctggccaccccctcaaccaccaccccaatcataggctaatgacggtggttgaagacatttaattgagattggcaatt

SEQ ID NO: 21 - DNA, 1383 bp, Feline sapelovirus
atcgtgtctacatcgcaatcaccaagacctgttaaccttaacacgaggacaactttgaggccttcagtgttctatgatgtcttcaagggggagaaggaacccgcagccctgcacatcagggacaggcgccttgaggtagacttagagaaagctatgttctcgaagtataagggtaaccttgacattgagtttcccccagagttgtccattgcggtggaccagtatgtggagcagatcagaccactgctgccgccggatctaaccgaaccattaccattggaggacgtggtctatggcattgagaaccttgagggccttgatctgaacacctcagcgggatacccttacgttaccatgggcatcaggaagaaagatttaatccctgagcgtggacagccactagataccttggtggaggccctcgacctccacggatacggccacccctacgtgacatatctcaaagatgagctgaggcccatagagaaggtcaagcaaggcaagacacgtttgattgagtgtagtagcctaaatgacaccattagattcaagacattctatggtaggttcatgcaggtcttccaccgcaatcctggcactataacaggtagtgctgtgggttgcaaccctgatgagcactggtcccaattctaccatgaattcaagcaggagccaataatggcatttgattatagcaattacgacgcttccctccatcccatctggttcgaagcactcaagctggtctttaggaaattaggctaccaagaggatagtctcaagctcattgaccacgtctgcttctccaaacatatttacaaatcaactttatatgaggtggagggaggtatgccatctggatgttctggcacatcggtcctcaactccatagtaaataacctcattatcaaaactttggtgctgagaacttataagggcattgatttggaccaactgaagatcctggcctacggggacgatgttatagtgacatacccgttccagctcgacgcctcgatcgtagcggatgagggaaggtcttttggcctgactatgacgccaccagataaaacatccacttttaatgagaccacctgggatacggtaaccttcctcaagcggaggttcgtcccagatgaacagtttcccttcctaattcacccagtctttcccatgtctgagatctacgagtccatccgctggactcggaacccacaacagaccaatgaacatgtggactcgctctgtcgattagcctggcactgcggggaacaggattacaatgactttgtcgccaaggtgcgctccgtgccggtggggcgggctctctcgctcccaacctaccgtgtcctgcgtgctttttggttagacctcttctaaaca